(19) United States
(12) Patent Application Publication
(10) Pub. No.: US 2021/0041454 A1
(43) Pub. Date: Feb. 11, 2021

(54) METHODS FOR PEPTIDE MASS SPECTROMETRY FRAGMENTATION PREDICTION

(71) Applicants: Immatics US, Inc., Houston, TX (US); Immatics Biotechnologies GmbH, Tuebingen (DE)

(72) Inventors: Chih-Chiang TSOU, Pearland, TX (US); Jens FRITSCHE, Dusslingen (DE); Toni WEINSCHENK, Aichwald (DE); Julian MUELLER, Tuebingen (DE)

(21) Appl. No.: 16/988,290

(22) Filed: Aug. 7, 2020

Related U.S. Application Data
(60) Provisional application No. 62/884,893, filed on Aug. 9, 2019.

(30) Foreign Application Priority Data
Aug. 9, 2019 (DE) 10 2019 121 600.1

(51) Int. Cl. G01N 33/68; G01N 30/88

(52) U.S. Cl. CPC G01N 33/6848; G01N 2030/027; G01N 30/88

(57) ABSTRACT

The present disclosure relates to methods of improved identification of peptides, for example, antigenic peptides. In particular, the present disclosure relates to methods of more accurately identifying human leukocyte antigen (HLA) peptides by utilizing classification systems. The disclosure also provides for utilizing the described methods for the field of personalized cancer therapies, such as adoptive cellular therapy (ACT).

### CROSS REFERENCE TO RELATED APPLICATIONS

This instant application claims priority to U.S. Provisional application No. 62/884,893, filed on Aug. 9, 2019, and German Patent Application number 10 2019 121 600.1, filed Aug. 9, 2019, the contents of each which are hereby incorporated by reference in their entireties.

### REFERENCE TO SEQUENCE LISTING SUBMITTED ELECTRONICALLY

The official copy of the sequence listing is submitted electronically via EFS-Web as an ASCII formatted sequence listing with a file named “3000011-014001_Seq_Listing_ST25.txt”, created on Aug. 7, 2020 and having a size of 3,686 bytes and is filed concurrently with the specification. The sequence listing contained in this ASCII formatted document is part of the specification and is herein incorporated by reference in its entirety.

### BACKGROUND

### Field

The present disclosure relates to methods of improved identification of peptides, for example, antigenic peptides. In particular, the present disclosure relates to methods of more accurately identifying human leukocyte antigen (HLA) peptides by utilizing methods and algorithms described herein. In an aspect, the disclosure provides for utilizing the described methods for the field of personalized cancer therapies, such as adoptive cellular therapy (ACT).

### Background

The rich repertoire of peptides presented by HLA class I (HLA-I) and HLA class II (HLA-II) complexes, referred to as the immunopeptidome, reflects the health state of a cell. HLA-bound peptides derived from cancer-specific and mutated proteins, pathogens and self-peptides in case of autoimmunity, may serve as targets for T-cell recognition. Clinical efficacy of immune checkpoint blockade therapies has led to discovery of immunogenic T-cell epitopes that mediate disease control or improved survival for development of personalized medicine.

Identification of immunopeptidome may rely on mass spectrometry by querying experimental mass spectra against theoretical fragment ions generated from peptide sequences. However, theoretical fragment ions may not represent the real peptide fragment spectra well enough, therefore identification may be severely limited. There have been developments toward in silico prediction of mass spectrometry peptide fragmentation, i.e. peptide mass spectrum prediction, however, none of these prediction algorithms showed sufficient results for applications of HLA immunopeptidome peptides.

Given the importance in drug discovery and next generation therapeutics, there remains a need to improve HLA peptide fragmentation prediction and associated peptide identification accuracy.

### BRIEF SUMMARY

In an aspect, the disclosure relates to methods for identifying one or more peptides including:
(a) analyzing one or more tissue samples by mass spectrometry (MS),
(b) acquiring experimental mass spectra from one or more peptides, for example antigenic peptides, bound to HLA-subtypes in the one or more tissue samples,
(c) generating a peptide spectrum match (PSM) of the one or more peptides by comparing the acquired mass spectra to peptide theoretical spectra,
(d) producing a matched spectral library or database of peptides, for example antigenic peptides, based on steps (a), (b), and (c),
(e) inputting the spectral library or database of peptides into an algorithm, for example a deep learning algorithm, to produce a spectral library of predicted peptide fragmentation spectra.

In another aspect, the disclosure relates to methods for identifying one or more peptides including:
(a) obtaining one or more tumor samples from an individual,
(b) acquiring mass spectrometry spectra for one or more peptides, for example antigenic peptides, bound to HLA-subtypes in the tumor sample,
(c) comparing the mass spectrometry data spectra to peptide theoretical spectra located in one or more public or non-public databases,
(d) generating a peptide spectrum match (PSM) of the one or more peptides,
(e) producing a matched spectral library or database of peptides, for example antigenic peptides, based on steps (a)-(d),
(g) inputting the spectral library or database of peptides into an algorithm, for example a deep learning algorithm, to produce a library of predicted peptide fragmentation spectra.

In an aspect, after inputting the library or database of peptides into a deep learning algorithm to produce a library of predicted peptide fragmentation spectra, the method includes matching the predicted peptide fragmentation spectra generated from the algorithm to the corresponding peptide spectrum match (PSM). Mass spectrometry (MS) generates a signal (spectrum) of values (m/z, intensity) related to the presence of a biomolecule with a certain mass-to-charge ratio (m/z), and abundance (intensity) in the sample. In addition to mass spectrometry data spectra, the methods described herein may further use retention time data.

In yet another aspect, the disclosure relates to methods for identifying peptides including:
(a) determining HLA-subtypes of an individual,
(b) obtaining at least one tumor tissue sample and corresponding healthy tissue sample from an individual,
(c) identifying one or more peptides, for example antigenic peptides, bound to HLA-subtypes in the tumor sample by mass spectrometry (MS),
(d) generating experimental spectra from the mass spectrometry data;
(e) comparing the mass spectrometry experimental spectra to spectra found in one or more public or non-public databases,
(f) generating a peptide spectrum match (PSM) of the one or more antigenic peptides,
(g) producing a spectral library or database of peptides,
(h) using a deep learning algorithm to generate a peptide mass spectrometry fragmentation prediction model using a portion of the peptide data found in the database or library as training data and subsequently testing the performance of the prediction model by a separate portion of peptide data found in the database of library,
(i) using the prediction model to generate predicted spectrum to identify peptides.

In a preferred aspect, the peptides to be identified are HLA-associated peptides.

In an aspect, the disclosure relates to methods for identifying antigenic peptides, including:
(a) obtaining at least one tumor tissue sample and corresponding healthy tissue sample from an individual,
(b) identifying one or more antigenic peptides bound to HLA-subtypes in a tumor tissue sample by mass spectrometry (MS), to produce one or more experimental peptide fragmentation spectra of the one or more antigenic peptides;
(c) comparing experimental peptide fragmentation spectra to those found in public and/or non-public databases;
(d) estimation of false discovery rate (FDR);
(e) generation of a peptide spectrum match (PSM);
(f) inputting the data generated by the experimental mass spectrometry methodology into a deep learning algorithm to train a peptide fragmentation prediction model;
(g) developing predicted peptide spectrum; and
(h) identifying one or more antigenic peptides, for example, previously unknown antigenic peptides.

In yet another aspect, the disclosure provides for methods of identifying antigenic peptides for use to induce a CTL immune response in an individual comprising:
(a) determining HLA-subtypes of an individual,
(b) obtaining a tumor sample and a corresponding healthy tissue sample from the individual,
(c) identifying one or more genes that are overexpressed in the tumor sample relative to the healthy tissue sample using microarray analysis, RNAseq, or RT-PCR,
(d) identifying one or more antigenic peptides bound to the HLA-subtypes in the tumor sample by mass spectrometry (MS) to produce peptide fragmentation spectra of the one or more antigenic peptides,
(e) inputting an HLA peptide sequence database into a deep learning algorithm to produce a library of predicted peptide fragmentation spectra corresponding to the input HLA peptide sequences,
(f) matching the peptide fragmentation spectra with the library of predicted peptide fragmentation spectra,
(g) identifying the sequences of the one or more antigenic peptides, when the peptide fragmentation spectra match the predicted peptide fragmentation spectra that correspond to the HLA peptide sequences,
(h) selecting only the antigenic peptides identified in step g), which are encoded by the one or more genes identified in step c), and
(i) synthesizing the antigenic peptides selected in step h), wherein the CTL immune response is induced in the individual.

In one embodiment, a method of identifying one of more antigenic peptides comprises:
(a) acquiring mass spectrometry spectra for one or more antigenic peptides;
(b) acquiring retention time data for the one or more antigenic peptides;
(c) comparing the mass spectrometry data spectra to peptide theoretical spectra located in one or more public or non-public databases,
(d) generating a peptide spectrum match (PSM) of the one or more antigenic peptides,
(e) producing a matched spectral library or database of antigenic peptides based on steps (a)-(d),
(f) using a deep learning algorithm to train at least 80% of the peptide data located in the database or spectral library and testing the balance of the peptide data located in the database or library thereby producing a peptide prediction model to generate predicted peptide spectrum;
(g) using the prediction model to identify one or more antigenic peptides.

In another embodiment, a method of identifying one of more antigenic peptides comprises:
(a) obtaining one or more tissue samples,
(b) acquiring mass spectrometry spectra for one or more antigenic peptides;
(c) acquiring retention time data for the one or more antigenic peptides;
(d) comparing the mass spectrometry data spectra to peptide theoretical spectra located in one or more public or non-public databases,
(e) generating a peptide spectrum match (PSM) of the one or more antigenic peptides,
(f) producing a matched spectral library or database of antigenic peptides based on steps (a)-(e),
(g) using a deep learning algorithm to train at least 80% of the peptide data located in the database or spectral library and testing the balance of the peptide data located in the database or library thereby producing a peptide prediction model to generate predicted peptide spectrum;
(h) using the prediction model to identify one or more antigenic peptides.

By utilizing methods described herein previously unknown peptides may be more accurately and efficiently be identified. In an aspect, prior to obtaining one or more tumor samples from an individual, the HLA-subtypes of the individual are determined.

In an aspect, methods described herein are capable of identifying peptides which were previously evaluated experimentally, for example by mass spectrometry but were not able to be identified due to similarity in terms of peptide fragmentation with other peptides. In such cases, methods described herein allow for better confidence and accuracy in identifying previously unidentified peptides.

In an aspect, the disclosure provides for methods of improving the confidence of peptide identification by mass spectrometry by using algorithms and methodology described herein.

In an aspect, methods described herein are used in a pre-clinical setting to better identify peptides. In another aspect, methods described herein are used in a clinical setting. In another aspect, methods herein are used in the field of personalized medicine, for example, in the adoptive cellular therapy (ACT) field.

In an aspect, tissue samples used for methods described herein are taken from tumor tissue and corresponding healthy tissue. In an aspect, the starting material for peptide identification is a non-cultured tissue or a cell line.

In another aspect, the mass spectrometry may include a tandem mass spectrometry (MS/MS).

In an aspect, methods described herein exhibit a better performance when predicting peptide fragmentation for HLA-associated peptides. In another aspect, methods described herein exhibit better performance when predicting peptide fragmentation for HLA-associated peptides as compared to identification of tryptic peptides by utilizing similar methodology.

In an aspect, after producing a prediction model by training and testing peptide data via an algorithm, utilizing the prediction model to generate predicted peptide tandem mass spectra. In an aspect, the predicted peptide tandem mass spectra can help identification of peptides which were previously not confidently identified by mass spectrometry alone.

In an aspect, the deep learning algorithm described herein is selected from pDeep (Zhou et al., Anal. Chem. 89, 12690-12697 (2017)), DeepMass (Tiwary et al., Nature Methods, 16:519-525 (2019)), and PROSIT (Gessulat et al., Nature Methods, 16:509-518 (2019), the disclosures of each of which are herein incorporated by reference in their entirety).

In an aspect, pDeep covers a deep learning-based method to predict the intensity distribution of product ions of a peptide. pDeep can work well in predicting not only higher-energy C-trap dissociation (HCD) spectra but also electron-transfer dissociation (ETD), and electron-transfer/higher-energy collision dissociation (EThcD) spectra. pDeep algorithm may allow for prediction of MS/MS spectra with peptide sequences without incorporating detailed fragmentation mechanisms into the model. For example, in pDeep, BiLSTM (bidirectional long short term memory (LSTM)), which has been successfully used to capture the bidirectional dependencies of sequential patterns in speech and natural languages, may be used to model the influences of both N- and C-terminal amino acids of each cleavage position on the site-specific peptide fragmentation. In addition, because the b/c ions depend on the N-terminal amino acids and the y/z ions depend on the C-terminal ones, to predict b/y/c/z ions together, both directions may be simultaneously considered. The BiLSTM-based pDeep can take the whole peptide as input, convert the different cleavage sites into feature vectors of different time-steps, and output the corresponding intensity of each peak.

In an aspect, HLA peptide data sets that contain more than about 100×10⁵ or about 200×10⁶ high-quality, high-resolution MS/MS spectra from Immatics XPRESIDENT® HLA peptidome data including HCD and collision-induced dissociation (CID) spectra may be used to train and test pDeep.

In another aspect, algorithms used herein are utilized to evaluate and improve databases or libraries that include over 70%, over 80%, over 85%, over 90%, or over 90% HLA peptides. In another aspect, algorithms herein are used to evaluate and improve databases or libraries that do not include more than 5%, more than 10%, more than 15%, more than 25%, or more than 30% tryptic peptides.

In another aspect, methods described herein include training of about 75%, about 80%, about 85%, about 90%, or about 95% of the data in the library or database and testing the remaining percentage for a total of 100% data. In an aspect, from about 70% to about 90% of the data is trained and the remainder of about 10% to about 30% of the data is tested, from about 80% to about 95% of the data is trained and the remainder of about 5% to about 20% of the data is tested, or from about 90% of the data is trained and the remainder of about 10% of the data is tested. In another aspect, the data that is trained and tested is experimentally identified data that is not in a public database.

In another aspect, methods described herein result in a higher spectral similarity score to the experimental spectra than other methodology, for example the prediction model built by public dataset of tryptic peptide spectra. See, ProteomeTools Dataset PXD004732; Zolg et al. Nat Methods (2017) 14: 259-262, the disclosure of which is herein incorporated by reference in its entirety. This results in more accurate identification of previously unidentified antigenic peptides.

In an aspect, methods described herein result in more accurate peptide fragmentation prediction performance. In another aspect, prediction performance is measured by dot product on a scale from 0 to 1, with 0 being the lowest score and 1 being the highest score. See, for example, Toprak et al. “Conserved Peptide Fragmentation as a Benchmarking Tool for Mass Spectrometers and a Discriminating Feature for Targeted Proteomics,” Mol Cell Proteomics (2014) 13(8): 2056-2071, the disclosure of which is herein incorporated by reference in its entirety. The prediction performance score measures the predicted spectra with the actual experimentally acquired spectra in order to gauge accuracy of peptide fragmentation prediction. Using this method, in an aspect, methods described herein provide for a prediction performance score of greater than about 0.9, 0.95, greater than about 0.955, greater than about 0.96, greater than about 0.965, greater than about 0.97, greater than about 0.975, or greater than about 0.98, about 0.90 to about 0.099, about 0.95 to about 0.98, or about 0.96 or about 0.99. Using the methods described herein, the prediction performance may be at about 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 0.991, 0.992, 0.993, 0.994, 0.995, 0.996, 0.997, 0.998, 0.999, or 1.00.

In another aspect, the disclosure provides for identifying one or more genes that are overexpressed in the tumor sample relative to the healthy tissue, for example, by utilizing microarray analysis, RT-PCR, or RNAseq in combination with methods described herein. For example, the expressed gene profile of a tumor sample may be compared to the same healthy tissue and analyzed by means of a classifier described herein.

In an aspect, methods described herein are capable of determining the presentation or expression of a given peptide in a specific tumor. In another aspect, a binder, such as a T-cell, TCR, bi-specific molecule, and/or antibody may be created against the identified peptide.

In an aspect, methods described herein are capable of determining whether the identified peptide is tumor specific. In another aspect, the tumor is a cancer tumor and associated with one or more of the following cancer types: hepatocellular carcinoma (HCC), colorectal carcinoma (CRC), glioblastoma (GB), gastric cancer (GC), esophageal cancer, non-small cell lung cancer (NSCLC), pancreatic cancer (PC), renal cell carcinoma (RCC), benign prostate hyperplasia (BPH), prostate cancer (PCA), ovarian cancer (OC), melanoma, breast cancer (BRCA), chronic lymphocytic leukemia (CLL), Merkel cell carcinoma (MCC), small cell lung cancer (SCLC), Non-Hodgkin lymphoma (NHL), acute myeloid leukemia (AML), gallbladder cancer and cholangiocarcinoma (GBC, CCC), urinary bladder cancer (UBC), and uterine cancer (UEC).

Peptides identified by the present methodology are capable of being used in a method for treating cancer. In another embodiment, the peptides identified by the methods described herein is an antigenic peptide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an exemplary process for MS/MS spectra analysis to identify the peptide sequences of HLA peptides in accordance with one embodiment of the disclosure.

FIG. 2 shows an example of peptide fragmentation for the analysis of a peptide sequence APNDFNLK (SEQ ID NO: 1) in accordance with one embodiment of the disclosure.

FIG. 3 shows an example of MS/MS spectrum of the peptide shown in FIG. 2 (SEQ ID NO: 1) in accordance with one embodiment of the disclosure.

FIG. 4 shows an example of peptide spectrum match between a predicted spectrum of a peptide sequence YLLPAIVHI (SEQ ID NO: 2) and an experimental spectrum in accordance with one embodiment of the disclosure.

FIG. 5 shows the correlation efficiency of prediction models using a deep learning algorithm in accordance with one embodiment of the disclosure.

FIG. 6 shows prediction models using a deep learning algorithm in accordance with one embodiment of the disclosure. IM spectra denote the HLA peptide MS/MS spectra acquired by Immatics XPRESIDENT® HLA peptidome platform. PT spectra denote the MS/MS spectra download from ProteomeTools Dataset PXD004732.

FIG. 7 shows a method of benchmarking a fragmentation prediction model with one embodiment of the disclosure including comparing experimental spectra of a peptide generated by MS/MS to the predicted spectra generated by a prediction model trained by a peptide spectra database. Technical variation, which is measured by comparing MS/MS spectra identified as a same peptide, is considered as the upper bound performance of any prediction model could achieve.

FIG. 8 shows correlation between experimental HLA peptide spectra and predicted spectra obtained from two different prediction models, one trained by CID 35 Immatics XPRESIDENT® HLA peptide spectra, and the other one trained by CID spectra from ProteomeTools Dataset in accordance with one embodiment of the disclosure.

FIG. 9 shows correlation between experimental tryptic peptide spectra and predicted spectra obtained from two different prediction models, and the other one trained by CID 35 Immatics XPRESIDENT® HLA peptide spectra, and another trained by CID spectra from ProteomeTools Dataset in accordance with another embodiment of the disclosure.

FIG. 10 shows correlation between experimental HLA peptide spectra and predicted spectra obtained from two different prediction models, one trained by HCD 28 Immatics XPRESIDENT® HLA peptide spectra, and the other one trained by HCD spectra from ProteomeTools Dataset in accordance with another embodiment of the disclosure.

FIG. 11 shows correlation between experimental tryptic peptide spectra and predicted spectra obtained from two different prediction models, one trained by HCD 28 Immatics XPRESIDENT® HLA peptide spectra, and the other one trained by HCD spectra from ProteomeTools Dataset in accordance with another embodiment of the disclosure.

FIG. 12 shows a method of building fragmentation prediction model using MS/MS spectra from tissue samples according to an aspect of the invention.

FIG. 13 shows an exemplary methodology according to an aspect of the invention. Exemplary target sequences: ISLLDAQSR (SEQ ID NO: 3), VVEELCPTPE (SEQ ID NO: 4), LLLQWCWE (SEQ ID NO: 5), CDVVSNTI (SEQ ID NO: 6). Exemplary decoy sequences: RSQADLLSI (SEQ ID NO: 7), EPTPCLEEVV (SEQ ID NO: 8), EWCWQLLL (SEQ ID NO: 9), ITNSVVDC (SEQ ID NO: 10). Exemplary peptide matches: VSVVDLTNT (SEQ ID NO: 11), VVEELCEGM (SEQ ID NO: 12), DLLLQWCWEN (SEQ ID NO: 13), ECDVVTIIAE (SEQ ID NO: 14), GDAVIDALN (SEQ ID NO: 15), SYLFCMEAE (SEQ ID NO: 16).

FIG. 14 shows a Dot Product comparison between Immatics-pDeep (HCD) (an embodiment of the system and method described herein) against Prosit pretrained model (HCD 25) and Prosit pretrain model (HCD 27) The comparison was done only for HCD model because the Prosit model is limited to HCD.

FIGS. 15A-B show a Dot Product analysis of two very similar peptides: KLLEVQILE (SEQ ID NO: 17) (FIG. 15B) and QLLEKVIEL (SEQ ID NO: 18) (FIG. 15A) which are difficult to distinguish using conventional methods. Using the Immatics-pDeep (HCD) (an embodiment of the system and method described herein) computed the predicted spectrum QLLEKVIEL (SEQ ID NO: 18) is 0.927.

FIG. 16 depicts a Dot Product showing the Dot Product scores computed by the true peptides with high false peptide incidents, 485 peptide pairs where SEQUEST was not able to clearly differentiae one from the other. The Immatics-pDeep (HCD) (an embodiment of the system and method described herein) showed an unexpected improvement in the prediction of sequence for peptides that have a high incidence of false positives.

FIG. 17 shows that the prediction model accurately predicts uRT (universal retention time) of HLA peptides. The average of prediction error (Actual-predicted) uRT is 0.061 and standard deviation of prediction error is 1.35.

FIG. 18 depicts the ROC curves by different database search rescoring approaches. Baseline: Comet database search. IMApDeep MSMS: Database rescoring by the fragmentation data prediction model built using pDeep algorithm and Immatics HLA mass spectrometry data. IMApDeep MSMS-NL: Database rescoring by the fragmentation data prediction model built using pDeep algorithm and Immatics HLA mass spectrometry data including neutral loss fragments. IMAProsit MSMS: Database rescoring by the fragmentation data prediction model built using Prosit algorithm and Immatics HLA mass spectrometry data. IMAProsit MSMS-NL: Database rescoring by the fragmentation data prediction model built using Prosit algorithm and Immatics HLA mass spectrometry data including neutral loss fragments. IMAProsit RT: Database rescoring by the peptide retention time prediction model built using Prosit algorithm and Immatics HLA mass spectrometry data. IMApDeep+IMAProsit: Database rescoring by combination of the peptide fragmentation data (built using pDeep algorithm) and retention time prediction models (built using Prosit algorithm) which is built using Immatics HLA mass spectrometry data as training data. This combination of mass spectra data and retention time shows an unexpected improvement in HLA peptide identification compared to Comet search engine (Baseline) using the methods described herein. “Rescored” refers to the rescoring configuration that received the best results: FDR=0.001: 1185 PSMs in Baseline vs. 15477 PSMs in Rescored (Relative Increase: 13.06); FDR=0.01: 22519 PSMs in Baseline vs. 31596 PSMs in Rescored (Relative Increase: 1.40); and FDR=0.05: 28413 PSMs in Baseline vs. 34410 PSMs in Rescored (Relative Increase: 1.21).

### DETAILED DESCRIPTION

### Improved Methods and Systems for Identification of HLA Peptides

The disclosure provides for methods of improving identification of peptides. In an aspect, the identified peptides are used in personalized medicine, such as cancer treatments and adoptive cellular therapy (ACT). In another aspect, methods described herein are improved over conventional peptide identification methods by resulting in less false positives of off-target peptide identification.

“AdaBoost,” as used herein, refers broadly to a bagging method that iteratively fits CARTs re-weighting observations by the errors made at the previous iteration.

“Antigenic peptides,” as used herein, may refer broadly to a peptide between about 5 and 14 amino acids that is capable of triggering a T-cell immune response, preferably protein fragments that bind to a MHC molecule to form a peptide-MHC complex. Antigenic peptides may comprise HLA peptides. The term “peptide” is used herein to designate a series of amino acid residues, connected one to the other by peptide bonds between the alpha-amino and carbonyl groups of the adjacent amino acids. The peptides are preferably 9 amino acids in length but can be as short as 8 amino acids in length, and as long as 10, 11, or 12, or longer, and in case of MHC class II peptides (elongated variants of the peptides of the invention) they can be as long as 13, 14, 15, 16, 17, 18, 19 or 20 or more amino acids in length.

“Classifier,” as used herein, refers broadly to a machine learning algorithm such as support vector machine(s), AdaBoost classifier(s), penalized logistic regression, elastic nets, regression tree system(s), gradient tree boosting system(s), naive Bayes classifier(s), neural nets, Bayesian neural nets, k-nearest neighbor classifier(s), Deep Learning systems, and random forests. This invention contemplates methods using any of the listed classifiers, as well as use of more than one of the classifiers in combination.

“Classification and Regression Trees (CART),” as used herein, refers broadly to a method to create decision trees based on recursively partitioning a data space so as to optimize some metric, usually model performance

“Classification system,” as used herein, refers broadly to a machine learning system executing at least one classifier.

“CTL,” as used herein, refers broadly to cytotoxic T-lymphocytes, generally CD8+ T cells.

“Elastic Net,” as used herein, refers broadly to a method for performing linear regression with a constraint comprised of a linear combination of the L1 norm and L2 norm of the vector of regression coefficients.

“False Positive (FP)” and “False Positive Identification,” as used herein, refers broadly to an error in which the algorithm test result indicates the presence of a disease when the disease is actually absent.

“False Negative (FN),” as used herein, refers broadly to an error in which the algorithm test result indicates the absence of a disease when the disease is actually present.

“Genetic Algorithm,” as used herein, refers broadly to an algorithm that mimics genetic mutation used to optimize a function (e.g., model performance).

“HLA peptide,” as used herein, refers broadly to an antigenic peptide that is bound in a peptide-MHC complex and presented to a T-cell. HLA peptides are antigenic peptides.

“LASSO,” as used herein, refers broadly to a method for performing linear regression with a constraint on the L1 norm of the vector of regression coefficients.

“L1 Norm,” as used herein, is the sum of the absolute values of the elements of a vector.

“L2 Norm,” as used herein, is the square root of the sum of the squares of the elements of a vector.

“Negative Predictive Value (NPV),” as used herein, is the number of true negatives (TN) divided by the number of true negatives (TN) plus the number of false negatives (FP), TP/(TN+FN).

“Neural Net,” as used herein, refers broadly to a classification method that chains together perceptron-like objects to create a classifier.

“Performance score,” as used herein, refers broadly to the distances between predicted values and actual values in the training data. This is expressed as a number between 0-100%, with higher values indicating the predicted value is closer to the real value. Typically, a higher score means the model performs better.

“Positive Predictive Value (PPV),” is the number of true positives (TP) divided by the number of true positives (TP) plus the number of false positives (FP), TP/(TP+FP).

“Random Forest,” as used herein, refers broadly to a bagging method that fits CARTs based on samples from the dataset that the model is trained on.

“Retention time,” and “retention time data,” as used herein, refer broadly to a measure of the time taken for a peptide to pass through a liquid chromatography column. It is calculated as the time from injection to detection.

“Ridge Regression,” as used herein, refers broadly to a method for performing linear regression with a constraint on the L2 norm of the vector of regression coefficients.

“Spectra data” “Spectrum data,” as used herein, refers broadly to mass spectrometry data for a peptide. The spectra data may be “experimental spectrum data” or “experimental spectrum” where the data is generated by mass spectrometer. The spectra data may be “predicted spectra data” or “predicted spectrum” where the data is generated by machine learning systems (e.g., classifiers) using the peptide sequence.

“Standard of Deviation (SD),” as used herein, is the spread in individual data points (i.e., in a replicate group) to reflect the uncertainty of a single measurement.

“Subset,” as used herein, refer broadly to a proper subset and “superset” is a proper superset.

“Training Set,” as used herein, is the set of samples that are used to train and develop a machine learning system, such as an algorithm used in the method and systems described herein.

“Tryptic peptides,” as used herein, refers broadly to the peptides produced by a trypsin digest is used to cleave the proteins in a sample downstream to every lysine or arginine residue, except when followed by a proline residue. A collection of tryptic peptides may comprise antigenic and HLA peptides.

“True Negative (TN),” as used herein, is the algorithm test result indicates that a peptide is not an antigenic when the peptide is actually antigenic.

“True Positive (TP),” as used herein, is the algorithm test result indicates that a peptide is antigenic when the peptide is actually antigenic.

“Validation Set,” as used herein, refers broadly to the set of samples that are blinded and used to confirm the functionality of the algorithm used in the method and systems described herein. This is also known as the Blind Set.

### Improved High Throughput Identification of HLA Peptides

Advantages of the methods of present disclosure may include improving high throughput identification of HLA peptides and providing less false positive as compared with experimental procedures alone, such as mass spectrometry and public database utilization. The methods described herein combine the experimental identification of peptides with algorithms capable of taking that data and training and testing the data to optimize peptide discover accuracy. For example, the methods described herein may utilize a classifier to identify HLA peptides, e.g., antigen peptides that trigger a T-cell response, from a data set on a peptide. Thus, methods of the present disclosure may provide a more efficient way, e.g., time reducing and specificity increasing, to select top HLA peptides to be formulated in personalized medicine by selecting fewer HLA peptides for validation with higher confidence.

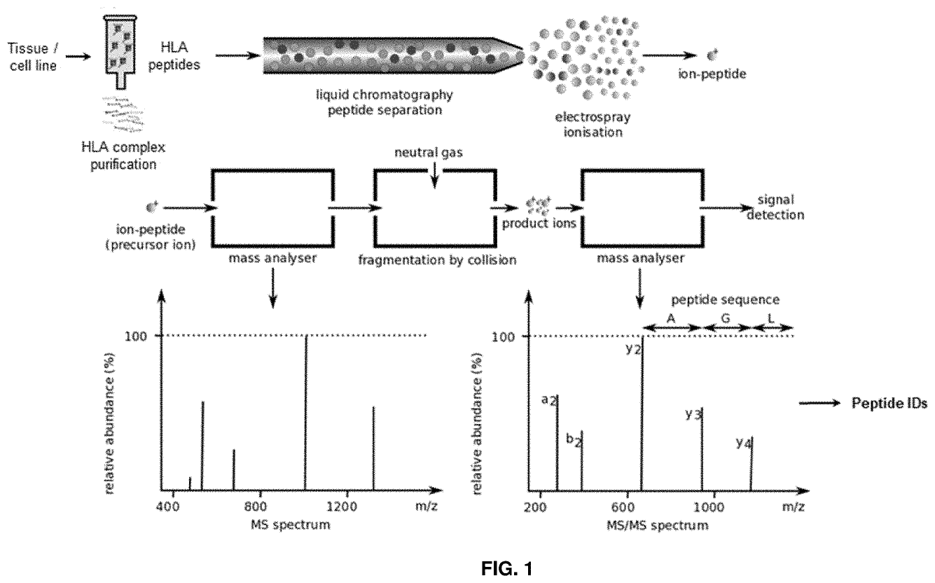

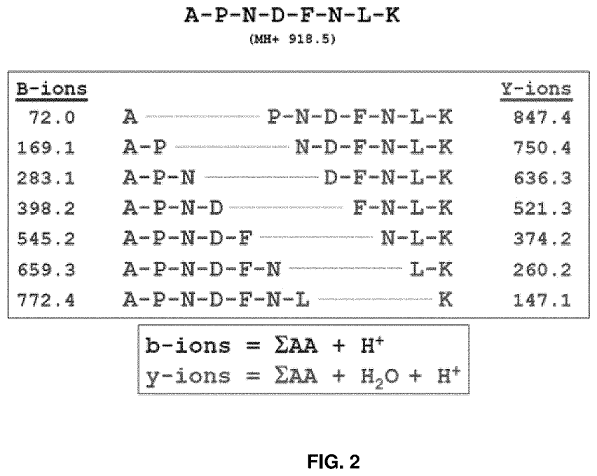

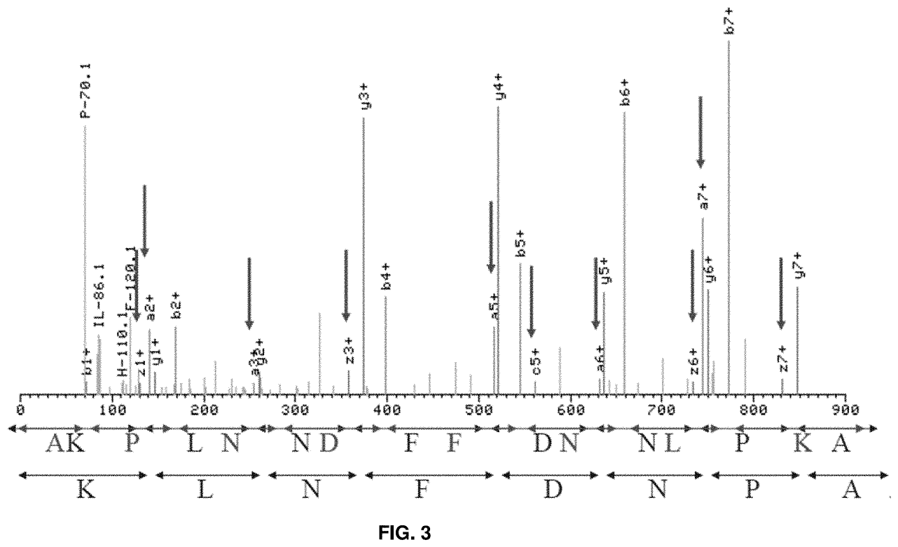

Mass spectrometry (MS) is a tool capable of identifying naturally (in vivo) presented HLA peptides in human cell lines, tumor tissues and bodily fluids, such as plasma. Mass spectrometry (MS) generates a signal (spectrum) of values (m/z, intensity) related to the presence of a biomolecule with a certain mass-to-charge ratio (m/z), and abundance (intensity) in the sample. The mass spectrometry data spectra may further comprises retention time data. FIG. 1 shows immunopeptidomics based on immunoaffinity purification of HLA complexes from mild detergent solubilized lysates, followed by extraction of the HLA peptides. The extracted peptides can then be separated by chromatography and directly injected into a mass spectrometer, such as tandem mass spectrometry (MS/MS). In MS/MS, parent or precursor ions generated from a sample may be selected by a first mass filter/analyzer and can then be passed to a collision cell, in which they are fragmented, such as b-ions and y-ions shown in FIG. 1, by collisions with neutral gas molecules to yield daughter or product ions. The fragment or daughter ions can then be mass analyzed by a second mass filter/analyzer and the resulting fragment or daughter ion spectra, such as that shown in FIG. 2, can be used to determine the structure and hence identify the parent or precursor ion. The methods may also comprise using retention time data for training the classification systems described herein.

In aspects, methods described herein improve the identification accuracy and/or determination of an antigenic peptide as its data acquired by the methods described in, for example, Philippe Hupé et al., “Mass spectrometry protocol”, Computational Systems Biology of Cancer Chapman & Hall/CRC Mathematical & Computational Biology, 2012, the disclosure of which is herein incorporated by reference in its entirety. The antigenic peptide data may comprise mass spectrum data, retention time, and combinations thereof.

In another aspect, peptide data acquired from the method of, for example FIG. 1, were assembled into a database or library and inputted into an algorithm, for example a deep learning algorithm, to produce a library of predicted peptide fragmentation spectra. In one embodiment, peptide data acquired from the method of, for example FIG. 1, are assembled into a database and processed by a classifier to produce a library of predicted peptide fragmentation spectra. These methods may further comprise using retention time data in the methodology.

### Peptides

In an aspect, peptides identified and evaluated by methods described herein are from 7 to 14 amino acids, 8 to 12 amino acids, 8 to 11 amino acids, 9 to 11 amino acids, 8 to 10 amino acids, less than 14, less than 12, less than 10, or 7, 8, 9, 10, or 11 amino acids in length. In one embodiment, the peptides are 5, 6, 7, 8, 9, 10, 11, 12, 13, or 14 amino acids in length. The peptide may be about 5-14 amino acids in length, about 6-12 amino acids in length, about 7-12 amino acids in length, or about 6-11 amino acids in length.

In an aspect, methods described herein include analysis of only antigenic peptides (HLA associated peptides). In an aspect, methods described are not used to identify and/or evaluate tryptic peptides. In another aspect, methods described herein more accurately identify antigenic peptides over tryptic peptides.

Trypsin is the “gold standard” for protein digestion to generate peptides for “shotgun proteomics,” e.g., a large-scale, non-specific approach. Trypsin is a serine protease that cleaves proteins with an average size of 700-1500 Daltons, which is in the ideal range for MS. Laskay et al. (2013) J Proteome Res. 12(12): 5558-69, the disclosure of which is herein incorporated by reference in its entirety. Trypsin cleaves selectively at the carboxyl side of arginine and lysine residues, except for positions where there is a proline residue on the carboxyl side of the cleave site. Trypsin is highly active and tolerant of several additives. The C-terminal arginine and lysine peptides are charged, making them detectable by MS.

Tryptic peptides, a mixture of peptides produced by digestion with trypsin, may contain antigenic peptides and HLA peptides, but this is not a specific process. The generation of tryptic peptides is driven by trypsin's specificity and not the potential antigenicity of a peptide. This leads to a large percentage of the peptides generated by a trypsin digestion being non-antigenic, e.g., of no use in T-cell therapies. Additionally, the trypsin digest eliminates potential HLA peptides of great value that may contain a lysine or arginine residue in the middle of the epitope.

The method described herein uses unconventional methods to better identify potential HLA peptides for use in T-cell therapies. Instead of performing a trypsin digest to produce a library of random peptides that are run through a mass spectroscopy, random, computer generated peptide fragments are generated from the parent protein and this library of fragments is processed by a classifier to identify antigenic peptides, in particular, HLA peptides. For example, the methods described herein may identify HLA peptides with a lysine or arginine residue in the epitope which would be destroyed by a trypsin digest. Further, the use of retention time data allows for further discrimination of different peptides, for example highly similar peptides, e.g., peptides that could not be distinguished using standard techniques.

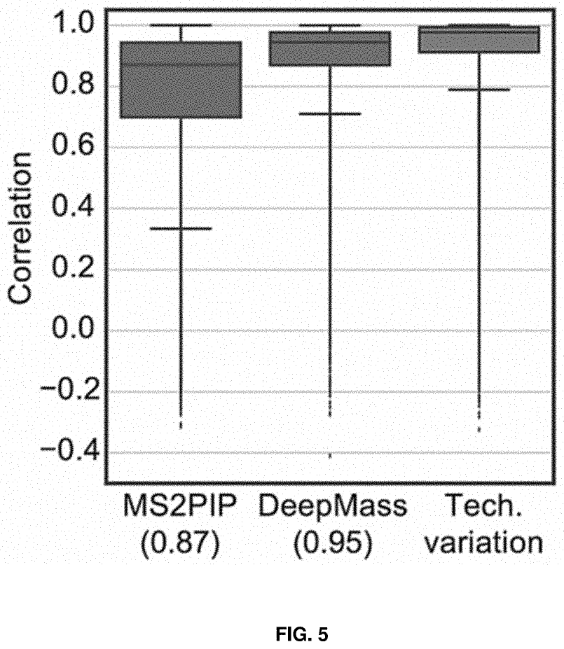

In an aspect, the methods described herein exhibit a prediction performance score (correlation) of antigenic peptide identification that is about 2% or less, about 5% or less, about 7% or less, about 10% or less, about 15% or less, about 20% or less, or about 25% or less, about 5% top about 25% less, about 5% to about 15% or less, or about 10% to about 25% or less, relative to the experimentally measured technical variation. In an embodiment, the performance score of the methods described herein is greater than 80%. For example, the performance score may be about 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%. The performance score may between about 90% and 95%, 92% and 98%, or 91% and 99%. See, for example, FIGS. 5 and 8-11.

In an aspect, methods described herein have a prediction performance score of about 3% or more, about 5% or more, about 7% or more, about 10% or more, about 15% or more, about 20% or more, about 25% or more, about 30% or more, about 35% or more, about 10% to about 75%, about 5% to about 50%, about 10% to about 40%, about 10% to about 30%, about 10% to about 25%, or about 10% to about 20% closer to the measured technical variation as compared to a convention identification model, for example, the PT model or mass spectrometry analysis without training and testing data as described herein.

In an aspect, methods described herein exhibit a higher predicted score when identifying HLA-associated peptides as compared to identification of tryptic peptides. In an aspect, methods described herein more accurately predict peptide matches related to the PT method. In a non-limiting aspect, a characteristic of methodologies described herein is a better correlation score when identifying antigenic peptides and a lower score when evaluating tryptic peptides.

In an aspect, the library or database described herein includes over about 60%, over about 70%, over about 80%, over about 85%, over about 90%, or over about 95% antigenic peptides. In another aspect, the library or database described herein includes about 60% to about 95%, about 60% to about 80%, about 70% to about 90%, about 80% to about 90%, or about 85% to about 95% antigenic peptides. For example, the database of peptides generated by the methods described herein may comprise about 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% antigenic peptides. The database may comprise between about 90% and 95%, 92% and 98%, or 91% and 99% antigenic peptides. Further, the database of peptides generated by the methods described herein may comprise about 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% HLA peptides. The database may comprise between about 90% and 95%, 92% and 98%, or 91% and 99% HLA peptides.

In an aspect, the library or database described herein contains less than about 30%, less than about 25%, less than about 20%, less than about 15%, less than about 10%, or less than about 5% tryptic peptides. In yet another aspect, the library or database described herein contains less than about 30%, less than about 25%, less than about 20%, less than about 15%, less than about 10%, or less than about 5% tryptic peptides. For example, the database of peptides generated by the methods described herein may comprise less than 30% non-antigenic peptides, e.g., peptides which do not elicit an immune response. The database of peptides may comprise less than 30%, 25%, 20%, 15%, 10%, 5%, 4%, 3%, 2%, or 1% non-antigenic peptides. The methods described herein produce an unexpected result of a nearly pure antigenic peptide library including antigenic peptides that could not be identified using the conventional trypsin digest-MS methods.

### Peptide Mutations

In an aspect, methods described herein are capable of identifying peptides with one, two, or three mutations relative to a base sequence. In some aspects, the mutation is a conservative substitution or mutation. In another aspect, methods described herein are capable of identifying peptides with a great specificity and higher prediction performance score as compared to methods wherein a machine learning system, e.g., Deep Learning, has not been used.

In an aspect, conservative substitutions may include those, which are described by Dayhoff in “The Atlas of Protein Sequence and Structure. Vol. 5”, Natl. Biomedical Research. For example, in an aspect, amino acids, which belong to one of the following groups, can be exchanged for one another, thus, constituting a conservative exchange: Group 1: alanine (A), proline (P), glycine (G), asparagine (N), serine (S), threonine (T); Group 2: cysteine (C), serine (S), tyrosine (Y), threonine (T); Group 3: valine (V), isoleucine (I), leucine (L), methionine (M), alanine (A), phenylalanine (F); Group 4: lysine (K), arginine (R), histidine (H); Group 5: phenylalanine (F), tyrosine (Y), tryptophan (W), histidine (H); and Group 6: aspartic acid (D), glutamic acid (E). In an aspect, a conservative amino acid substitution may be selected from the following of T→A, G→A, A→I, T→V, A→M, T→I, A→V, T→G, and/or T→S. See, e.g., Johnson & Petersen (2001) “A Critical View on Conservative Mutations.” Protein Engineering, Design and Selection 14(6): 397-402; Ng & Henikoff (2001) Genome Res. 11: 863-874, the contents of which are herein incorporated by reference in their entirety.

In an aspect, a conservative amino acid substitution may include the substitution of an amino acid by another amino acid of the same class, for example, (1) nonpolar: Ala, Val, Leu, Ile, Pro, Met, Phe, Trp; (2) uncharged polar: Gly, Ser, Thr, Cys, Tyr, Asn, Gln; (3) acidic: Asp, Glu; and (4) basic: Lys, Arg, His. Other conservative amino acid substitutions may also be made as follows: (1) aromatic: Phe, Tyr, His; (2) proton donor: Asn, Gln, Lys, Arg, His, Trp; and (3) proton acceptor: Glu, Asp, Thr, Ser, Tyr, Asn, Gln. See, for example, U.S. Pat. No. 10,106,805 and Yampolsky & Stoltzfus “The Exchangeability of Amino Acids in Proteins.” Genetics 170: 1459-1472, the disclosures of each of which are herein incorporated by reference in its entirety.

### Peptide Fragmentation Profiles

Accurate modeling of peptide fragmentation is an important factor for the development of robust scoring functions for peptide-spectrum matches, which are the cornerstone of MS/MS-based identification algorithms Unfortunately, peptide fragmentation is a complex process that can involve several competing chemical pathways, which makes it difficult to develop generative probabilistic models that describe it accurately.

By providing for greater resolution, methods described herein are capable of identifying peptides that were previously difficult to identify peptide sequences with a greater specificity and accuracy. For example, potentially useful epitopes that would be destroyed in a trypsin digest. This results in less false positive identifications. In an aspect, methods described herein are capable of identifying peptides with the same amino acid sequence but in a different configuration. In an aspect, amino acid positions are adjacent or further down the amino acid chain.

As a non-limiting example, conventional peptide identification methods have trouble identifying closely related sequences. By combining confirmed experimental data with algorithms, such as pDeep, the methods described herein have the ability to identify previously un-identified peptides. For example, by classifying a library of peptides it is possible to identify previously unidentified HLA antigens, including HLA antigens that were overlooked or destroyed by conventional methods.

In an aspect, the peptide may be known, but association with a specific tumor or cancer-type unknown. In such instances, methods described herein have the ability to identify and determine the sequence identity and tumor association. For example, a classifier may be used to identify and determine the sequence identity and tumor association.

In another aspect, the mass spectrometry may be performed in a data independent acquisition (DIA) mode or in a data dependent acquisition (DDA) mode.

In another aspect, the HLA peptide sequence database may contain at least 2×10⁶ PSMs generated by collision-induced dissociation (CID), surface-induced dissociation (SID), electron-capture dissociation (ECD), higher-energy C-trap dissociation (HCD), electron-transfer dissociation (ETD), negative electron-transfer dissociation (NETD), electron-detachment dissociation (EDD), infrared multiphoton dissociation (IRMPD), blackbody infrared radiative dissociation (BIRD), electron-transfer/higher-energy collision dissociation (EThcD), or electron-transfer and collision-induced dissociation (ETCID).

In another aspect, the PSMs may have a false discovery rate (FDR) of less than 0.05 or less than 0.10.

Peptide spectrum matches (PSMs) herein refer to the identified peptide sequences (peptide spectrum matches) for the protein, including those redundantly identified. The number of PSMs is the total number of identified peptide spectra matched for the protein. The PSM value may be higher than the number of peptides identified for high-scoring proteins because peptides may be identified repeatedly.

Data-independent acquisition (DIA) herein refers to a method of molecular structure determination, in which all ions within a selected m/z range may be fragmented and analyzed in a second stage of tandem mass spectrometry. Tandem mass spectra may be acquired either by fragmenting all ions that enter the mass spectrometer at a given time (called broadband DIA) or by sequentially isolating and fragmenting ranges of m/z.

Data-dependent acquisition (DDA) herein refers to, in contrast to DIA, a mode of data collection in tandem mass spectrometry, in which a fixed number of precursor ions whose m/z values recorded in a survey scan may be selected using predetermined rules and are subjected to a second stage of mass selection in an MS/MS analysis.

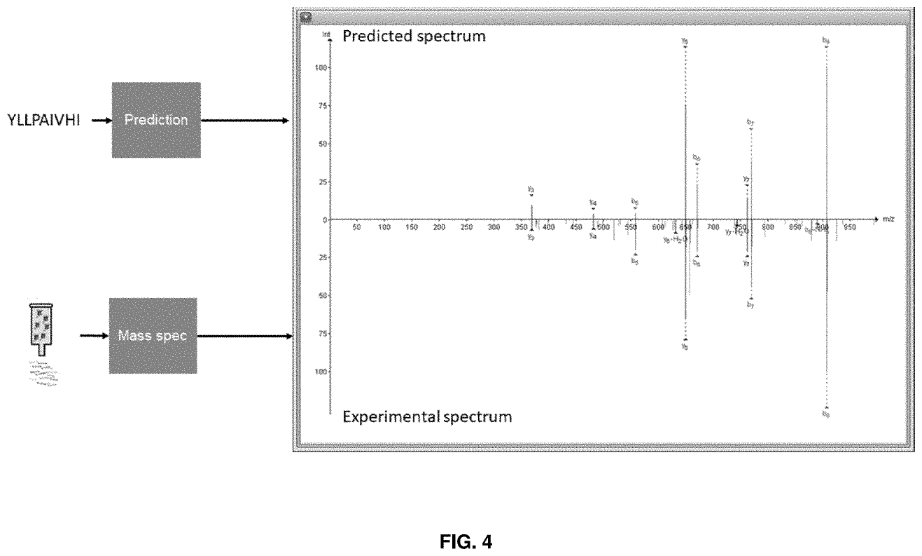

Deep learning is a class of machine learning algorithms that use multiple layers to progressively extract higher level features from raw input. Deep learning algorithms may be capable of much more accurate prediction performance for applications in computer vision and image/video/audio recognition. Its multiple layers provide deeper description for different perspectives. Take image processing as an example, lower layers may identify edges, while higher layer may describe human-meaningful items such as digits/letters or faces. Deep learning may also be applied for mass spectrometry data analysis, including de novo sequencing, retention time prediction and peptide fragmentation prediction. As an example, shown in FIG. 4, a deep learning prediction model can successfully predict spectrum peak intensities, i.e. peptide fragmentation pattern of a peptide.

Predicted spectrum herein refers to a peptide fragmentation spectrum generated by machine learning algorithms such as classifiers.

### Methods for Predicting Peptide Fragment Structure

Matching fragment ion spectra to peptide sequences is relevant to the identification, quantification and subsequent biological interpretation. One method is database searching, in which a fragmentation spectrum is matched to theoretical spectra for candidate peptides generated in silico. Some search engines, include, for example, Andromeda (Cox et al. J Proteome Res. 10(4):1794-805, 2011, the contents of which are incorporated by reference in their entirety), score peptide spectrum matches (PSMs) on the presence of fragment ions but largely disregard fragment ion intensities or information regarding which fragment ions may be experimentally observed.

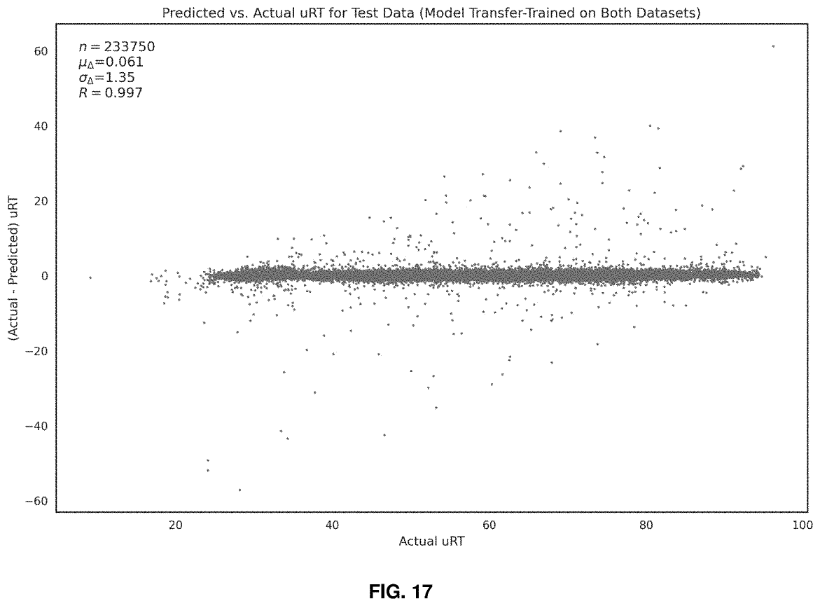

Spectral library searching may be a complementary approach, in which intensities of peptide fragment ions from experimental spectra may be correlated to library spectra, typically constructed from previous peptide identification data. Spectral libraries are commonly used for the analysis of targeted or data-independent acquisition (DIA) experiments. In DIA, additional information, such as peptide retention time, may be useful in improving confident peptide identification and quantification. Retention time of a peptide during liquid chromatography represents a relative hydrophobicity which can also be employed to differentiate one peptide from another. The classification methods and systems described herein show an unexpected improvement in the prediction of retention time. The inventors trained the classifiers using Prosit's algorithm and Immatics' immunopeptidome databases. FIG. 17 shows that the classification system described herein accurately predicts uRT (universal retention time) of HLA peptides. The average of prediction error (Actual-predicted) uRT is 0.061 and standard deviation of prediction error is 1.35.

### Classification Systems

The invention relates to, among other things, characterizing peptides based on MS data, preferably MS/MS data comprising experimental peptide spectrum data sets. The experimental peptide spectrum data sets may be propriety or accessed from publicly available databases.

DeepMass prediction model, as described, for example, in Tiwary et al. Nat Methods (2019) 16(6): 519-525, the content of which is incorporated by reference in its entirety, can achieve a better correlation (0.95) between experimental spectra and predicted spectra than MS2PIP prediction model (0.87), as also indicated by the closer correlation of the former to the technical variation than that of the latter.

MassAnalyzer tool is a deductive physicochemical model of peptide fragmentation. Parameters in this model may be optimized on a dataset containing 8900 MS/MS spectra. Zhang Anal. Chem. (2004) 76: 3908-3922; Zhang Anal. Chem. (2005) 77: 6364-6373, the content of which is incorporated by reference in its entirety.

PeptideART is based on feed-forward neural network representations. It can implement an ensemble of neural networks, in which each network models the most important fragment ion peak intensities in one multi-output feed-forward neural network. This method models the (normalized) peak intensities directly. Arnold et al. Pacific Symposium on Biocomputing. (2006) pages 219-230, the content of which is incorporated by reference in its entirety.

PepNovo uses a boosting algorithm (in the context of ranking) to take advantage of experimental MS/MS data to create models for predicting the intensity ranks of a peptide's fragment ion peaks based only on the peptide's amino acid sequence without having to fully understand the complex dynamics of peptide fragmentation. Frank, J Proteome Res. (2009) 8: 2226-2240, the content of which is incorporated by reference in its entirety.

MS2PIP is a dataset of merged PSMs and presents an inductive learning approach for peak intensity regression that exploits information contained in this large number of PSMs. This approach uses the non-linear decision tree representation for training the peak intensity prediction models. Degroeve et al. Bioinformatics (2013) 29: 3199-3203, the content of which is incorporated by reference in its entirety.

U.S. Patent Application Publication No. 2008/0275651, the content of which is incorporated by reference in its entirety, describes a method of inferring presence of at least one protein in a sample, including entering a peptide training data set into a statistical inference model, e.g., a machine learning system, training the statistical inference model with the peptide training data set, determining predicted detectability of at least one peptide present in the sample with the trained statistical inference model, and inferring the presence of the at least one protein in the sample based upon the determined predicted detectability.

The machine learning systems, MassAnalyzer, PeptideART, PepNovo, and MS2PIP may be used in the system and method described herein. However, these systems have limitations and inaccuracies that are overcome by the methods described herein. For example, Deep Learning systems, including but not limited to pDeep, DeepMass, and PROSIT have shown unexpected improvements in the prediction of peptide sequences over conventional machine learning systems.

The classification systems used herein may include computer executable software, firmware, hardware, or combinations thereof. For example, the classification systems may include reference to a processor and supporting data storage. Further, the classification systems may be implemented across multiple devices or other components local or remote to one another. The classification systems may be implemented in a centralized system, or as a distributed system for additional scalability. Moreover, any reference to software may include non-transitory computer readable media that when executed on a computer, causes the computer to perform a series of steps.

The classification systems described herein may include data storage such as network accessible storage, local storage, remote storage, or a combination thereof. Data storage may utilize a redundant array of inexpensive disks (“RAID”), tape, disk, a storage area network (“SAN”), an internet small computer systems interface (“iSCSI”) SAN, a Fibre Channel SAN, a common Internet File System (“CIFS”), network attached storage (“NAS”), a network file system (“NFS”), or other computer accessible storage. The data storage may be a database, such as an Oracle database, a Microsoft SQL Server database, a DB2 database, a MySQL database, a Sybase database, an object oriented database, a hierarchical database, Cloud-based database, public database, or other database. Data storage may utilize flat file structures for storage of data. Exemplary embodiments used two Tesla K80 NVIDIA GPUs, each with 4992 CUDA cores and large amounts of GB of memory (e.g., over 11 GB) to train the deep learning algorithms

In the first step, a classifier is used to describe a pre-determined set of data. This is the “learning step” and is carried out on “training” data.

The training database is a computer-implemented store of data reflecting a plurality of peptide spectrum data for a plurality of peptides association with a classification with respect to antigen characterization of each respective peptide. The peptide spectrum data may comprise experimental peptide spectrum data, predicted peptide spectrum data, or a combination thereof. The format of the stored data may be as a flat file, database, table, or any other retrievable data storage format known in the art. The test data may be stored as a plurality of vectors, each vector corresponding to an individual peptide, each vector including a plurality of peptide spectrum data measures for a plurality of experimental peptide spectrum data together with a classification with respect to antigenicity characterization of the peptide. The vector may further comprise retention time data measures for a plurality of experimental peptide retention data together with a classification with respect to the antigenicity characterisation of the peptide. Typically, each vector contains an entry for each peptide spectrum data measure in the plurality of peptide spectrum data measures. The entry may further comprise retention time data. The training database may be linked to a network, such as the internet, such that its contents may be retrieved remotely by authorized entities (e.g., human users or computer programs). Alternately, the training database may be located in a network-isolated computer. Further, the training database may be Cloud-based, including proprietary and public databases containing peptide spectrum data (e.g., experimental, predicted, and combinations thereof) for antigenic peptides useful in immunoncology methods.

In the second step, which is optional, the classifier is applied in a “validation” database and various measures of accuracy, including sensitivity and specificity, are observed. In an exemplary embodiment, only a portion of the training database is used for the learning step, and the remaining portion of the training database is used as the validation database. In the third step, peptide spectrum data measures from a subject are submitted to the classification system, which outputs a calculated classification (e.g., characterization of a peptide as antigenic) for the subject. Additionally, retention time data may also be used.

There are many possible classifiers that could be used on the data. Machine and deep learning classifiers include but are not limited to AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, naive Bayes classifiers, neural nets, penalized logistic regression, Random Forests, ridge regression, support vector machines, or an ensemble thereof, may be used to classify the data. See e.g., Han & Kamber (2006) Chapter 6, Data Mining, Concepts and Techniques, 2nd Ed. Elsevier: Amsterdam. As described herein, any classifier or combination of classifiers (e.g., ensemble) may be used in a classification system. As discussed herein, the data may be used to train a classifier.

In an embodiment, the classifier is a Deep Learning algorithm. Machine learning is a subset of artificial intelligence that uses a machine's ability to take a set of data and learn about the information it is processing by changing the algorithm as data is being processed. Deep learning is a subset of machine learning that utilizes artificial neural networks inspired by the workings on the human brain. For example, the deep learning architecture may be multilayer perceptron neural network (MLPNN), backpropagation, Convolutional Neural Network (CNN), Recurrent Neural Network (RNN), Long Short-Term Memory (LSTM), Generative Adversarial Network (GAN), Restricted Boltzmann Machine (RBM), pDeep, DeepMass, PROSIT, Deep Belief Network (DBN), or an ensemble thereof. Deep Learning systems have shown a surprisingly high prediction performance for prediction models of peptide spectra data. Accordingly, the Deep Learning systems have an unexpected advantage in successfully predicting spectrum peak intensities, e.g., peptide fragmentation patterns, over other methods.

### Classification Trees

A classification tree is an easily interpretable classifier with built in feature selection. A classification tree recursively splits the data space in such a way so as to maximize the proportion of observations from one class in each subspace.

The process of recursively splitting the data space creates a binary tree with a condition that is tested at each vertex. A new observation is classified by following the branches of the tree until a leaf is reached. At each leaf, a probability is assigned to the observation that it belongs to a given class. The class with the highest probability is the one to which the new observation is classified.

Classification trees are essentially a decision tree whose attributes are framed in the language of statistics. They are highly flexible but very noisy (the variance of the error is large compared to other methods).

Tools for implementing classification tree are available, by way of non-limiting example, for the statistical software computing language and environment, R. For example, the R package “tree,” version 1.0-28, includes tools for creating, processing and utilizing classification trees. Examples of Classification Trees include but are not limited to Random Forest. See also Kamiński et al. (2017) “A framework for sensitivity analysis of decision trees.” Central European Journal of Operations Research. 26(1): 135-159; Karimi & Hamilton (2011) “Generation and Interpretation of Temporal Decision Rules”, International Journal of Computer Information Systems and Industrial Management Applications, Volume 3, the content of which is incorporated by reference in its entirety.

### Random Forests

Classification trees are typically noisy. Random forests attempt to reduce this noise by taking the average of many trees. The result is a classifier whose error has reduced variance compared to a classification tree. Methods of building a Random Forest classifier, including software, are known in the art. Prinzie & Poel (2007) “Random Multiclass Classification: Generalizing Random Forests to Random MNL and Random NB”. Database and Expert Systems Applications. Lecture Notes in Computer Science. 4653; Denisko & Hoffman (2018) “Classification and interaction in random forests”. PNAS 115(8): 1690-1692, the content of which is incorporated by reference in its entirety.

To classify a new observation using the random forest, classify the new observation using each classification tree in the random forest. The class to which the new observation is classified most often amongst the classification trees is the class to which the random forest classifies the new observation. Random forests reduce many of the problems found in classification trees but at the trade off of interpretability.

Tools for implementing random forests as discussed herein are available, by way of non-limiting example, for the statistical software computing language and environment, R. For example, the R package “random Forest,” version 4.6-2, includes tools for creating, processing and utilizing random forests.

### AdaBoost (Adaptive Boosting)

AdaBoost provides a way to classify each of n subjects into two or more categories based on one k-dimensional vector (called a k-tuple) of measurements per subject. AdaBoost takes a series of “weak” classifiers that have poor, though better than random, predictive performance and combines them to create a superior classifier. The weak classifiers that AdaBoost uses are classification and regression trees (CARTs). CARTs recursively partition the dataspace into regions in which all new observations that lie within that region are assigned a certain category label. AdaBoost builds a series of CARTs based on weighted versions of the dataset whose weights depend on the performance of the classifier at the previous iteration. Han & Kamber (2006) Data Mining, Concepts and Techniques, 2^{nd} Ed. Elsevier: Amsterdam, the content of which is incorporated by reference in its entirety. AdaBoost technically works only when there are two categories to which the observation can belong. For g>2 categories, (g/2) models must be created that classify observations as belonging to a group of not. The results from these models can then be combined to predict the group membership of the particular observation. Predictive performance in this context is defined as the proportion of observations misclassified.

### Convolutional Neural Network

Convolutional Neural Network (CNN or ConvNet) is a class of deep, feed-forward artificial neural networks, most commonly applied to analyzing visual imagery. CNNs use a variation of multilayer perceptrons designed to require minimal preprocessing. They are also known as shift invariant or space invariant artificial neural networks (SIANN), based on their shared-weights architecture and translation invariance characteristics. Convolutional networks were inspired by biological processes in that the connectivity pattern between neurons resembles the organization of the animal visual cortex. Individual cortical neurons respond to stimuli only in a restricted region of the visual field known as the receptive field. The receptive fields of different neurons partially overlap such that they cover the entire visual field. CNNs use relatively little pre-processing compared to other image classification algorithms This means that the network learns the filters that in traditional algorithms were hand-engineered. This independence from prior knowledge and human effort in feature design is a major advantage. LeCun and Bengio (1995) “Convolutional networks for images, speech, and time-series,” in Arbib (Ed.), The Handbook of Brain Theory and Neural Networks, MIT Press, the content of which is incorporated by reference in its entirety. Fully convolutional indicates that the neural network is composed of convolutional layers without any fully-connected layers or MLP usually found at the end of the network. Convolutional Neural Network is an example of Deep learning.

### Support Vector Machines

Support vector machines (SVMs) are recognized in the art. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction. Vapnik (1998) Statistical Learning Theory; Vapnik “An overview of statistical learning theory” IEEE Transactions on Neural Networks 10(5): 988-999 (1999) the content of which is incorporated by reference in its entirety. Any new observation is then classified as belonging to any one of the categories of interest, based where the observation lies in relation to the hyperplane. When more than two categories are considered, the process is carried out pairwise for all of the categories and those results combined to create a rule to discriminate between all the categories.

In an exemplary embodiment, a kernel function known as the Gaussian Radial Basis Function (RBF) is used. Vapnik, 1998. The RBF is often used when no a priori knowledge is available with which to choose from a number of other defined kernel functions such as the polynomial or sigmoid kernels. Han et al. Data Mining: Concepts and Techniques Morgan Kaufman 3^{rd} Ed. (2012). The RBF projects the original space into a new space of infinite dimension. A discussion of this subject and its implementation in the R statistical language can be found in Karatzoglou et al. “Support Vector Machines in R” Journal of Statistical Software 15(9) (2006), the content of which is incorporated by reference in its entirety. All SVM statistical computations described herein were performed using the statistical software programming language and environment R 2.10.0. SVMs were fitted using the ksvm( ) function in the kernlab package.

Cristianini, N., & Shawe-Taylor, J. (2000). An Introduction to Support Vector Machines and Other Kernel-based Learning Methods. Cambridge: Cambridge University Press provides some notation for support vector machines, as well as an overview of the method by which they discriminate between observations from multiple groups.

Other suitable Kernel functions include, but are not limited to, linear kernels, radial basis Kernels, polynomial Kernels, uniform Kernels, triangle Kernels, Epanechnikov Kernels, quartic (biweight) Kernels, tricube (triweight) Kernels, and cosine Kernels.

Support vector machines are one out of many possible classifiers that could be used on the data. By way of non-limiting example, and as discussed below, other methods such as naive Bayes classifiers, classification trees, k-nearest neighbor classifiers, etc. may be used on the same data used to train and verify the support vector machine. Naïve Bayes Classifier

The set of Bayes Classifiers are a set of classifiers based on Bayes' Theorem. See, e.g., Joyce (2003), Zalta, Edward N. (ed.), “Bayes' Theorem”, The Stanford Encyclopaedia of Philosophy (Spring 2019 Ed.), Metaphysics Research Lab, Stanford University, the content of which is incorporated by reference in its entirety.

All classifiers of this type seek to find the probability that an observation belongs to a class given the data for that observation. The class with the highest probability is the one to which each new observation is assigned. Theoretically, Bayes classifiers have the lowest error rates amongst the set of classifiers. In practice, this does not always occur due to violations of the assumptions made about the data when applying a Bayes classifier.

The naïve Bayes classifier is one example of a Bayes classifier. It simplifies the calculations of the probabilities used in classification by making the assumption that each class is independent of the other classes given the data.

Naïve Bayes classifiers are used in many prominent anti-spam filters due to the ease of implantation and speed of classification but have the drawback that the assumptions required are rarely met in practice.

Tools for implementing naive Bayes classifiers as discussed herein are available for the statistical software computing language and environment, R. For example, the R package “e1071,” version 1.5-25, includes tools for creating, processing and utilizing naive Bayes classifiers.

### Neural Nets

One way to think of a neural net is as a weighted directed graph where the edges and their weights represent the influence each vertex has on the others to which it is connected. There are two parts to a neural net: the input layer (formed by the data) and the output layer (the values, in this case classes, to be predicted). Between the input layer and the output layer is a network of hidden vertices. There may be, depending on the way the neural net is designed, several vertices between the input layer and the output layer.

Neural nets are widely used in artificial intelligence and data mining but there is the danger that the models the neural nets produce will over fit the data (i.e., the model will fit the current data very well but will not fit future data well). Tools for implementing neural nets as discussed herein are available for the statistical software computing language and environment, R. For example, the R package “e1071,” version 1.5-25, includes tools for creating, processing and utilizing neural nets.

k-Nearest Neighbor Classifiers (KNN)

The nearest neighbor classifiers are a subset of memory-based classifiers. These are classifiers that have to “remember” what is in the training set in order to classify a new observation. Nearest neighbor classifiers do not require a model to be fit.

To create a k-nearest neighbor (knn) classifier, the following steps are taken:
1. Calculate the distance from the observation to be classified to each observation in the training set. The distance can be calculated using any valid metric, though Euclidian and Mahalanobis distances are often used.
2. Count the number of observations amongst the k nearest observations that belong to each group. 3. The group that has the highest count is the group to which the new observation is assigned.

The Mahalanobis distance is a metric that takes into account the covariance between variables in the observations.

Nearest neighbor algorithms have problems dealing with categorical data due to the requirement that a distance be calculated between two points but that can be overcome by defining a distance arbitrarily between any two groups. This class of algorithm is also sensitive to changes in scale and metric. With these issues in mind, nearest neighbor algorithms can be very powerful, especially in large data sets.

Tools for implementing k-nearest neighbor classifiers as discussed herein are available for the statistical software computing language and environment, R. For example, the R package “e1071,” version 1.5-25, includes tools for creating, processing and utilizing k-nearest neighbor classifiers.

### Training Data

In another aspect, methods described herein include training of about 75%, about 80%, about 85%, about 90%, or about 95% of the data in the library or database and testing the remaining percentage for a total of 100% data. In an aspect, from about 70% to about 90% of the data is trained and the remainder of about 10% to about 30% of the data is tested, from about 80% to about 95% of the data is trained and the remainder of about 5% to about 20% of the data is tested, or from about 90% of the data is trained and the remainder of about 10% of the data is tested.

In an aspect, the database or library contains data from the analysis of over about 500, about 1000, over about 1500, over about 2000, over about 2500, or over about 3000 tissue samples, preferably tumor tissue samples. In an aspect, tumor tissue and healthy tissue from the same individual were analyzed.

In another aspect, the database or library contains over about 5000, over about 10,000, over about 15,000, over about 20,000, or over about 25,000 MS analysis on the collected tissue or tumor samples. In yet another aspect, the database or library contains over 1 million, 10 million, over 50 million, over 100 million, over 150 million, or over 200 million MS or MS/MS spectra. In a further aspect, the library or database includes only verified experimental data, for example from mass spectrometry or tandem mass spectrometry. In yet another aspect, the library or database does not include spectra and/or data that were theoretically prepared without the determination of peptide discovery or prevalence by analyzing patient tissue.

The training data may comprise mass spectrum data, retention time data, or combinations thereof.

### Methods of Classifying Data Using Classification System(s)

The invention provides for methods of classifying data (test data, e.g., peptide spectra data, retention time data) obtained from an individual. These methods involve preparing or obtaining training data, as well as evaluating test data obtained from an individual (as compared to the training data), using one of the classification systems including at least one classifier as described above. Preferred classification systems use classifiers such as, but not limited to, support vector machines (SVM), AdaBoost, penalized logistic regression, naive Bayes classifiers, classification trees, k-nearest neighbor classifiers, Deep Learning classifiers, neural nets, random forests, Fully Convolutional Networks (FCN), Convolutional Neural Networks (CNN), and/or an ensemble thereof. Deep Learning classifiers are a more preferred classification system. The classification system outputs a classification of the peptide based on the test data, e.g., peptide spectra data, retention time data, combinations thereof.

Particularly preferred for the present invention is an ensemble method used on a classification system, which combines multiple classifiers. For example, an ensemble method may include SVM, AdaBoost, penalized logistic regression, naive Bayes classifiers, classification trees, k-nearest neighbor classifiers, neural nets, Fully Convolutional Networks (FCN), Convolutional Neural Networks (CNN), Random Forests, Deep Learning, or any ensemble thereof, in order to make a prediction regarding peptide antigenicity (e.g., HLA peptide, antigenic peptide). The ensemble method was developed to take advantage of the benefits provided by each of the classifiers, and replicate measurements of each peptide spectrum data.

A method of classifying test data, the test data comprising predicted spectrum data for a peptide comprising: (a) accessing an electronically stored set of training data vectors, each training data vector or k-tuple representing an individual peptide and comprising peptide spectrum data for the respective peptide for each replicate, the training data vector further comprising a classification with respect to peptide characterization of each respective peptide; (b) training an electronic representation of a classifier or an ensemble of classifiers as described herein using the electronically stored set of training data vectors; (c) receiving test data comprising a plurality of peptide spectrum data for protein; (d) evaluating the test data using the electronic representation of the classifier and/or an ensemble of classifiers as described herein; and (e) outputting a classification of the peptide based on the evaluating step. The test data may further comprise retention time data.

In another embodiment, the invention provides a method of classifying test data, the test data comprising HLA peptide data comprising: (a) accessing an electronically stored set of training data vectors, each training data vector or k-tuple representing an individual human and comprising HLA peptide data for the respective human for each replicate, the training data further comprising a classification with respect to antigenicity of each respective HLA peptide; (b) using the electronically stored set of training data vectors to build a classifier and/or ensemble of classifiers; (c) receiving test data comprising a plurality of HLA peptide data for a human test subject; (d) evaluating the test data using the classifier(s); and (e) outputting a classification of the human test subject based on the evaluating step. Alternatively, all (or any combination of) the replicates may be averaged to produce a single value for each HLA peptide data for each subject. Outputting in accordance with this invention includes displaying information regarding the classification of the human test subject in an electronic display in human-readable form. The HLA peptide data may comprise peptide spectrum data, retention time, or combinations thereof.

The set of training vectors may comprise at least 20, 25, 30, 35, 50, 75, 100, 125, 150, or more vectors.

The test data may be any peptide information measures such as possible antigenic peptide sequence, retention time data, Mass Spectroscopy (MS) data, predicted MS spectral data for a peptide, or a combination thereof.

The data used to train a machine learning system, e.g., Deep Learning, may comprise data from tumors, including at least 5, 10, 15, 20, or 25 different indications, data from normal tissues, including at least about 5, 10, 15, 20, 25, 30, 35, 40, or 45 normal (tumor-free) tissues, or a combination thereof. In addition, the data used to train a machine learning system, e.g., Deep Learning, may comprise CID (Collision-induced dissociation) data, HCD (Higher-energy collisional dissociation) data, or a combination thereof.

It will be understood that the methods of classifying data may be used in any of the methods described herein. In particular, the methods of classifying data described herein may be used in methods for peptide characterization and identifying antigenic peptides, including HLA peptides, for use in immunoncology methods.

Particularly preferred for the present invention is an ensemble method used on a classification system, which combines multiple classifiers. For example, an ensemble method may include Support Vector Machine (SVM), AdaBoost, penalized logistic regression, naive Bayes classifiers, classification trees, k-nearest neighbor classifiers, neural nets, Deep Learning systems, Random Forests, or any combination thereof, in order to make a prediction regarding antigenicity of a peptide. In addition, the ensemble may be used to make a prediction regarding the association of a peptide with a type of cancer. The ensemble approach takes advantage of the benefits provided by each of the classifiers, and replicate measurements of each peptide.

### Computer-Implemented Methods

As used herein, the term “computer” is to be understood to include at least one hardware processor that uses at least one memory. The at least one memory may store a set of instructions. The instructions may be either permanently or temporarily stored in the memory or memories of the computer. The processor executes the instructions that are stored in the memory or memories in order to process data. The set of instructions may include various instructions that perform a particular task or tasks, such as those tasks described herein. Such a set of instructions for performing a particular task may be characterized as a program, software program, or simply software.

As noted above, the computer executes the instructions that are stored in the memory or memories to process data. This processing of data may be in response to commands by a user or users of the computer, in response to previous processing, in response to a request by another computer and/or any other input, for example.

The computer used to at least partially implement embodiments may be a general purpose computer. However, the computer may also utilize any of a wide variety of other technologies including a special purpose computer, a computer system including a microcomputer, mini-computer or mainframe for example, a programmed microprocessor, a micro-controller, a peripheral integrated circuit element, a CSIC (Customer Specific Integrated Circuit) or ASIC (Application Specific Integrated Circuit) or other integrated circuit, a logic circuit, a digital signal processor, a programmable logic device such as a FPGA, PLD, PLA or PAL, or any other device or arrangement of devices that is capable of implementing at least some of the steps of the processes of the invention.

It is appreciated that in order to practice the method of the invention, it is not necessary that the processors and/or the memories of the computer be physically located in the same geographical place. That is, each of the processors and the memories used by the computer may be located in geographically distinct locations and connected so as to communicate in any suitable manner Additionally, it is appreciated that each of the processor and/or the memory may be composed of different physical pieces of equipment. Accordingly, it is not necessary that the processor be one single piece of equipment in one location and that the memory be another single piece of equipment in another location. That is, it is contemplated, for example, that the processor may be two or more pieces of equipment in two different physical locations. The two or more distinct pieces of equipment may be connected in any suitable manner, such as a network. Additionally, the memory may include two or more portions of memory in two or more physical locations.

Various technologies may be used to provide communication between the various computers, processors and/or memories, as well as to allow the processors and/or the memories of the invention to communicate with any other entity; e.g., so as to obtain further instructions or to access and use remote memory stores, for example. Such technologies used to provide such communication might include a network, the Internet, Intranet, Extranet, LAN, an Ethernet, or any client server system that provides communication, for example. Such communications technologies may use any suitable protocol such as TCP/IP, UDP, or OSI, for example.

Further, it is appreciated that the computer instructions or set of instructions used in the implementation and operation of the invention are in a suitable form such that a computer may read the instructions.

In some embodiments, a variety of user interfaces may be utilized to allow a human user to interface with the computer or machines that are used to at least partially implement the embodiment. A user interface may be in the form of a dialogue screen. A user interface may also include any of a mouse, touch screen, keyboard, voice reader, voice recognizer, dialogue screen, menu box, list, checkbox, toggle switch, a pushbutton or any other device that allows a user to receive information regarding the operation of the computer as it processes a set of instructions and/or provide the computer with information. Accordingly, a user interface is any device that provides communication between a user and a computer. The information provided by the user to the computer through the user interface may be in the form of a command, a selection of data, or some other input, for example.

It is also contemplated that a user interface of the invention might interact, e.g., convey and receive information, with another computer, rather than a human user. Accordingly, the other computer might be characterized as a user. Further, it is contemplated that a user interface utilized in the system and method of the invention may interact partially with another computer or computers, while also interacting partially with a human user.

### Mass Spectroscopy

Experimental spectrum herein refers to a peptide spectrum generated by mass spectrometry. Complete mass spectrometry data may also include retention time of a mass spectrum, i.e. retention time data.

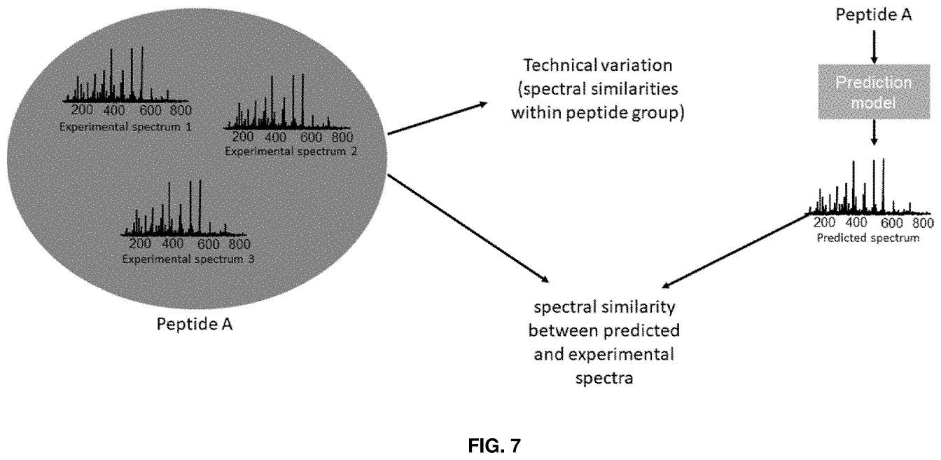

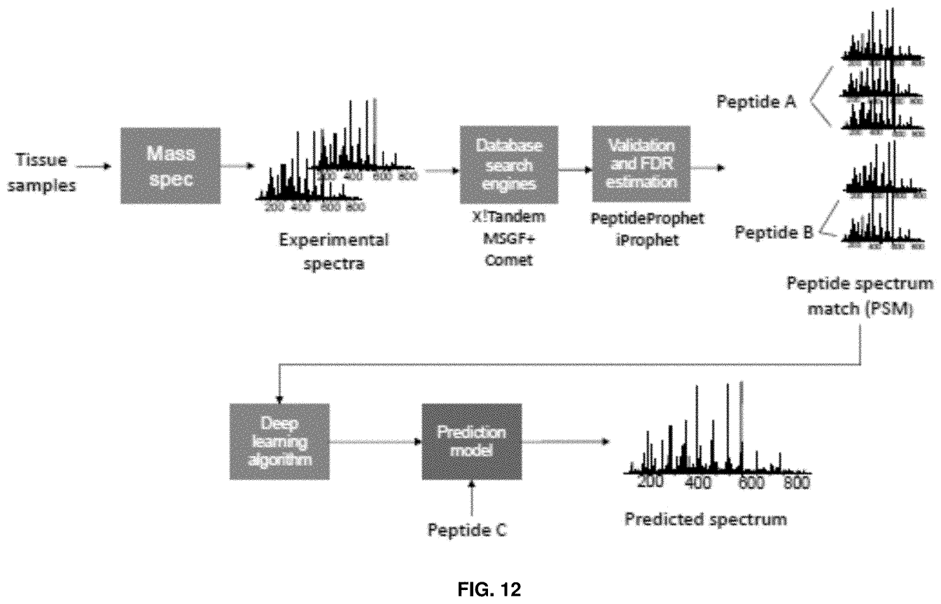

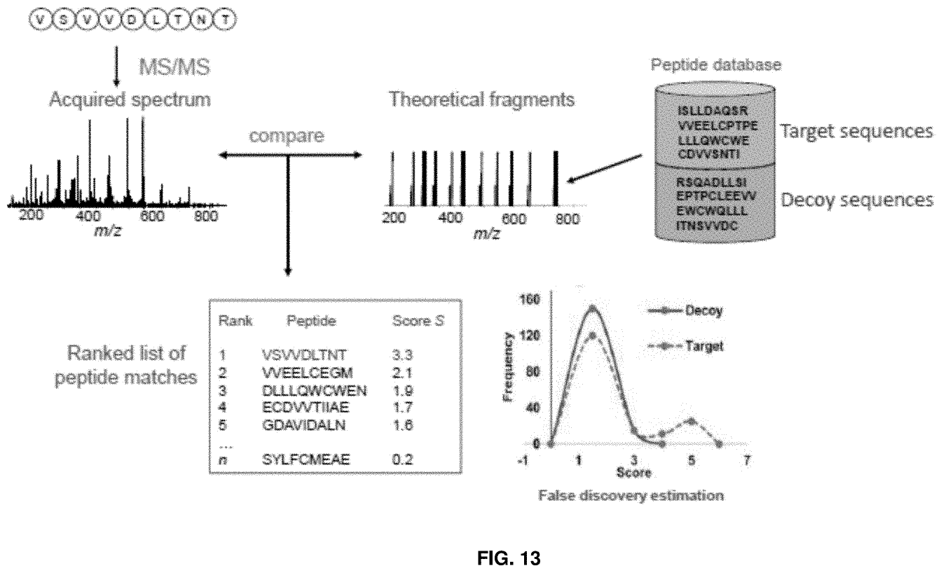

Technical variation herein refers to peptide spectra similarities between two replicate spectra from a same peptide, which represents the upper bound of peptide fragmentation prediction performance for any algorithm. For example, the technical variation is a given peptide A is determined by comparing the distinct experimental peptide spectra. See, for example, FIG. 7. The prediction model, which determines the predicted peptide spectrum as generated by methods described herein, can be compared to the experimental spectra to determine the accuracy of the prediction model. As set forth in FIG. 12, technical variation corresponds to the correlation between replicate experimental confirmation of peptide spectrum. As technical variation is based on actual experiments, such as confirmation of a peptide by mass spectrometry, and not on predicted confirmation of structure, the score is closer to 1 (highest) and theoretically should be higher than any predicted model. The closer a prediction model has a score closer to 1, the more accurate the prediction of peptide fragmentation pattern. Using the methods described herein, the prediction performance may be at about 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 0.991, 0.992, 0.993, 0.994, 0.995, 0.996, 0.997, 0.998, 0.999, or. The prediction performance may be between about 0.80 and 1.00, 0.85 and 0.95, 0.90 and 0.995, or 0.95 and 0.99.

Mass spectrometry and computational data analysis have transformed proteomics research and enabled proteome scale analysis of biological systems. In immunopeptidome, for example, HLA peptides may be subjected to an initial mass spectrometry analysis, designated MS1. The MS1 spectrum depicts the intensity of signal, which corresponds to the amount of the peptide present in the sample. Ions of interest in the MS1 spectrum may be selected and subjected to a second mass spectrometry event, induced by collision-induced dissociation (CID), which results in fragmentation of the selected peptide to yield a second mass spectrum (MS2) with sufficient information to permit identification of the peptide by comparison to available databases. The signal peaks in an MS/MS spectrum indicate the presence of a peptide fragment ion with a specific mass. The intensity of a signal peak is dependent on a number of factors: the abundance of the peptide in the sample, the efficiency of the cleavage that generated the fragment, the proteotypicity of the fragment ion and other factors related to the peptide and the machine that generated the MS2 spectrum. However, because such MS/MS analysis systems are limited by the ability of the instrument to detect a peptide of interest in the MS1 spectrum, only proteins of relatively high abundance may be detected. Furthermore, even if a particular protein is identified, the intensity of a particular m/z ratio in an MS1 spectrum may not permit quantitation absent some internal standard. The use of machine learning systems, as described herein, overcomes the short-comings in these methods by applying an unconventional steps to better characterize a peptide. The improvement in prediction using the machine learning systems was unexpected and lead to an improvement in the identification of antigenic peptides. For example, the peptides identified as antigenic using the methods described herein may be used in adoptive cellular therapies (ACT), including cytotoxic T-lymphocyte (CTL) applications. Additionally, the methods used herein may utilized retention time data. This was in contrast to the standard method of using mass spectroscopy which suffered from the afore-mentioned limitations.

In one embodiments, the mass spectrometer may include (a) an ion source selected from the group consisting of: (i) an Electrospray ionisation (“ESI”) ion source; (ii) an Atmospheric Pressure Photo Ionisation (“APPI”) ion source; (iii) an Atmospheric Pressure Chemical Ionisation (“APCI”) ion source; (iv) a Matrix Assisted Laser Desorption Ionisation (“MALDI”) ion source; (v) a Laser Desorption Ionisation (“LDI”) ion source; (vi) an Atmospheric Pressure Ionisation (“API”) ion source; (vii) a Desorption Ionisation on Silicon (“DIOS”) ion source; (viii) an Electron Impact (“EI”) ion source; (ix) a Chemical Ionisation (“CI”) ion source; (x) a Field Ionisation (“FI”) ion source; (xi) a Field Desorption (“FD”) ion source; (xii) an Inductively Coupled Plasma (“ICP”) ion source; (xiii) a Fast Atom Bombardment (“FAB”) ion source; (xiv) a Liquid Secondary Ion Mass Spectrometry (“LSIMS”) ion source; (xv) a Desorption Electrospray Ionisation (“DESI”) ion source; (xvi) a Nickel-63 radioactive ion source; (xvii) an Atmospheric Pressure Matrix Assisted Laser Desorption Ionisation ion source; (xviii) a Thermospray ion source; (xix) an Atmospheric Sampling Glow Discharge Ionisation (“ASGDI”) ion source; (xx) a Glow Discharge (“GD”) ion source; (xxi) an Impactor ion source; (xxii) a Direct Analysis in Real Time (“DART”) ion source; (xxiii) a Laserspray Ionisation (“LSI”) ion source; (xxiv) a Sonicspray Ionisation (“SSI”) ion source; (xxv) a Matrix Assisted Inlet Ionisation (“MAII”) ion source; (xxvi) a Solvent Assisted Inlet Ionisation (“SAII”) ion source; (xxvii) a Desorption Electrospray Ionisation (“DESI”) ion source; and (xxviii) a Laser Ablation Electrospray Ionisation (“LAESI”) ion source; and/or (b) one or more continuous or pulsed ion sources; and/or (c) one or more ion guides; and/or (d) one or more ion mobility separation devices and/or one or more Field Asymmetric Ion Mobility Spectrometer devices; and/or (e) one or more ion traps or one or more ion trapping regions; and/or (f) one or more collision, fragmentation or reaction cells selected from the group consisting of: (i) a Collisional Induced Dissociation (“CID”) fragmentation device; (ii) a Surface Induced Dissociation (“SID”) fragmentation device; (iii) an Electron Transfer Dissociation (“ETD”) fragmentation device; (iv) an Electron Capture Dissociation (“ECD”) fragmentation device; (v) an Electron Collision or Impact Dissociation fragmentation device; (vi) a Photo Induced Dissociation (“PID”) fragmentation device; (vii) a Laser Induced Dissociation fragmentation device; (viii) an infrared radiation induced dissociation device; (ix) an ultraviolet radiation induced dissociation device; (x) a nozzle-skimmer interface fragmentation device; (xi) an in-source fragmentation device; (xii) an in-source Collision Induced Dissociation fragmentation device; (xiii) a thermal or temperature source fragmentation device; (xiv) an electric field induced fragmentation device; (xv) a magnetic field induced fragmentation device; (xvi) an enzyme digestion or enzyme degradation fragmentation device; (xvii) an ion-ion reaction fragmentation device; (xviii) an ion-molecule reaction fragmentation device; (xix) an ion-atom reaction fragmentation device; (xx) an ion-metastable ion reaction fragmentation device; (xxi) an ion-metastable molecule reaction fragmentation device; (xxii) an ion-metastable atom reaction fragmentation device; (xxiii) an ion-ion reaction device for reacting ions to form adduct or product ions; (xxiv) an ion-molecule reaction device for reacting ions to form adduct or product ions; (xxv) an ion-atom reaction device for reacting ions to form adduct or product ions; (xxvi) an ion-metastable ion reaction device for reacting ions to form adduct or product ions; (xxvii) an ion-metastable molecule reaction device for reacting ions to form adduct or product ions; (xxviii) an ion-metastable atom reaction device for reacting ions to form adduct or product ions; and (xxix) an Electron Ionisation Dissociation (“EID”) fragmentation device; and/or (g) a mass analyzer selected from the group consisting of: (i) a quadrupole mass analyser; (ii) a 2D or linear quadrupole mass analyser; (iii) a Paul or 3D quadrupole mass analyser; (iv) a Penning trap mass analyser; (v) an ion trap mass analyser; (vi) a magnetic sector mass analyser; (vii) Ion Cyclotron Resonance (“ICR”) mass analyser; (viii) a Fourier Transform Ion Cyclotron Resonance (“FTICR”) mass analyser; (ix) an electrostatic mass analyser arranged to generate an electrostatic field having a quadro-logarithmic potential distribution; (x) a Fourier Transform electrostatic mass analyser; (xi) a Fourier Transform mass analyser; (xii) a Time of Flight mass analyser; (xiii) an orthogonal acceleration Time of Flight mass analyser; and (xiv) a linear acceleration Time of Flight mass analyser; and/or (h) one or more energy analyzers or electrostatic energy analyzers; and/or (i) one or more ion detectors; and/or (j) one or more mass filters selected from the group consisting of: (i) a quadrupole mass filter; (ii) a 2D or linear quadrupole ion trap; (iii) a Paul or 3D quadrupole ion trap; (iv) a Penning ion trap; (v) an ion trap; (vi) a magnetic sector mass filter; (vii) a Time of Flight mass filter; and (viii) a Wien filter; and/or (k) a device or ion gate for pulsing ions; and/or (l) a device for converting a substantially continuous ion beam into a pulsed ion beam.

In another embodiment, the mass spectrometer may further include: (i) a C-trap and a mass analyser comprising an outer barrel-like electrode and a coaxial inner spindle-like electrode that form an electrostatic field with a quadro-logarithmic potential distribution, wherein in a first mode of operation ions are transmitted to the C-trap and are then injected into the mass analyser and wherein in a second mode of operation ions are transmitted to the C-trap and then to a collision cell or Electron Transfer Dissociation device wherein at least some ions are fragmented into fragment ions, and wherein the fragment ions are then transmitted to the C-trap before being injected into the mass analyser; and/or (ii) a stacked ring ion guide comprising a plurality of electrodes each having an aperture through which ions are transmitted in use and wherein the spacing of the electrodes increases along the length of the ion path, and wherein the apertures in the electrodes in an upstream section of the ion guide have a first diameter and wherein the apertures in the electrodes in a downstream section of the ion guide have a second diameter which is smaller than the first diameter, and wherein opposite phases of an AC or RF voltage are applied, in use, to successive electrodes.

In another embodiment, the mass spectrometer may further include a device arranged and adapted to supply an AC or RF voltage to the electrodes. The AC or RF voltage optionally has an amplitude selected from the group consisting of: (i) about <50 V peak to peak; (ii) about 50-100 V peak to peak; (iii) about 100-150 V peak to peak; (iv) about 150-200 V peak to peak; (v) about 200-250 V peak to peak; (vi) about 250-300 V peak to peak; (vii) about 300-350 V peak to peak; (viii) about 350-400 V peak to peak; (ix) about 400-450 V peak to peak; (x) about 450-500 V peak to peak; and (xi) >about 500 V peak to peak. The AC or RF voltage may have a frequency selected from the group consisting of: (i) about 10.0 MHz.

In another embodiment, the mass spectrometer may also include a chromatography or other separation device upstream of an ion source. According to an embodiment the chromatography separation device comprises a liquid chromatography or gas chromatography device. According to another embodiment the separation device may comprise: (i) a Capillary Electrophoresis (“CE”) separation device; (ii) a Capillary Electrochromatography (“CEC”) separation device; (iii) a substantially rigid ceramic-based multilayer microfluidic substrate (“ceramic tile”) separation device; or (iv) a supercritical fluid chromatography separation device.

In another embodiment, analyte ions may be subjected to Electron Transfer Dissociation (“ETD”) fragmentation in an Electron Transfer Dissociation fragmentation device. Analyte ions may be caused to interact with ETD reagent ions within an ion guide or fragmentation device.

In another embodiment, in order to effect Electron Transfer Dissociation, either: (a) analyte ions are fragmented or are induced to dissociate and form product or fragment ions upon interacting with reagent ions; and/or (b) electrons are transferred from one or more reagent anions or negatively charged ions to one or more multiply charged analyte cations or positively charged ions whereupon at least some of the multiply charged analyte cations or positively charged ions are induced to dissociate and form product or fragment ions; and/or (c) analyte ions are fragmented or are induced to dissociate and form product or fragment ions upon interacting with neutral reagent gas molecules or atoms or a non-ionic reagent gas; and/or (d) electrons are transferred from one or more neutral, non-ionic or uncharged basic gases or vapors to one or more multiply charged analyte cations or positively charged ions whereupon at least some of the multiply charged analyte cations or positively charged ions are induced to dissociate and form product or fragment ions; and/or (e) electrons are transferred from one or more neutral, non-ionic or uncharged superbase reagent gases or vapors to one or more multiply charged analyte cations or positively charged ions whereupon at least some of the multiply charge analyte cations or positively charged ions are induced to dissociate and form product or fragment ions; and/or (f) electrons are transferred from one or more neutral, non-ionic or uncharged alkali metal gases or vapors to one or more multiply charged analyte cations or positively charged ions whereupon at least some of the multiply charged analyte cations or positively charged ions are induced to dissociate and form product or fragment ions; and/or (g) electrons are transferred from one or more neutral, non-ionic or uncharged gases, vapors or atoms to one or more multiply charged analyte cations or positively charged ions whereupon at least some of the multiply charged analyte cations or positively charged ions are induced to dissociate and form product or fragment ions, wherein the one or more neutral, non-ionic or uncharged gases, vapors or atoms are selected from the group consisting of: (i) sodium vapor or atoms; (ii) lithium vapor or atoms; (iii) potassium vapor or atoms; (iv) rubidium vapor or atoms; (v) cesium vapor or atoms; (vi) francium vapor or atoms; (vii) vapor or atoms; and (viii) magnesium vapor or atoms.

The multiply charged analyte cations or positively charged ions may include peptides, polypeptides, proteins or biomolecules.

In another embodiment, in order to effect Electron Transfer Dissociation: (a) the reagent anions or negatively charged ions are derived from a polyaromatic hydrocarbon or a substituted polyaromatic hydrocarbon; and/or (b) the reagent anions or negatively charged ions are derived from the group consisting of: (i) anthracene; (ii) 9,10 diphenyl-anthracene; (iii) naphthalene; (iv) fluorine; (v) phenanthrene; (vi) pyrene; (vii) fluoranthene; (viii) chrysene; (ix) triphenylene; (x) perylene; (xi) acridine; (xii) 2,2′ dipyridyl; (xiii) 2,2′ biquinoline; (xiv) 9-anthracenecarbonitrile; (xv) dibenzothiophene; (xvi) 1,10′-phenanthroline; (xvii) 9′ anthracenecarbonitrile; and (xviii) anthraquinone; and/or (c) the reagent ions or negatively charged ions comprise azobenzene anions or azobenzene radical anions.

In another embodiment, the process of Electron Transfer Dissociation fragmentation may include interacting analyte ions with reagent ions, wherein the reagent ions comprise dicyanobenzene, 4-nitrotoluene or azulene.

A chromatography detector may be provided wherein the chromatography detector may include either: a destructive chromatography detector optionally selected from the group consisting of (i) a Flame Ionization Detector (FID); (ii) an aerosol-based detector or Nano Quantity Analyte Detector (NQAD); (iii) a Flame Photometric Detector (FPD); (iv) an Atomic-Emission Detector (AED); (v) a Nitrogen Phosphorus Detector (NPD); and (vi) an Evaporative Light Scattering Detector (ELSD); or a non-destructive chromatography detector optionally selected from the group consisting of: (i) a fixed or variable wavelength UV detector; (ii) a Thermal Conductivity Detector (TCD); (iii) a fluorescence detector; (iv) an Electron Capture Detector (ECD); (v) a conductivity monitor; (vi) a Photoionization Detector (PID); (vii) a Refractive Index Detector (RID); (viii) a radio flow detector; and (ix) a chiral detector.

The mass spectrometer may be operated in various modes of operation including a mass spectrometry (“MS”) mode of operation, a tandem mass spectrometry (“MS/MS”) mode of operation, a mode of operation in which parent or precursor ions are alternatively fragmented or reacted so as to produce fragment or product ions, and not fragmented or reacted or fragmented or reacted to a lesser degree, a Multiple Reaction Monitoring (“MRM”) mode of operation, a Data Dependent Analysis (“DDA”) mode of operation, a Data Independent Analysis (“DIA”) mode of operation, a Quantification mode of operation or an Ion Mobility Spectrometry (“IMS”) mode of operation.

In the context of the present invention, one of the techniques and methods listed as follows may be preferably applied: a) A combination of any number of different mass spectrometry machines and mass spectrometry fragmentation techniques (e.g., collision-induced dissociation (CID), surface-induced dissociation (SID), electron-capture dissociation (ECD), Higher-energy C-trap dissociation (HCD), electron-transfer dissociation (ETD), negative electron-transfer dissociation (NETD), electron-detachment dissociation (EDD), infrared multiphoton dissociation (IRMPD), blackbody infrared radiative dissociation (BIRD), electron-transfer/higher-energy collision dissociation (EThcD), electron-transfer and collision-induced dissociation (ETCID)) or activation energies to allow for better sequence coverage of the peptide tandem MS (MS/MS) spectra; b) Mass spectrometry experiments in data dependent (DDA) as well as data independent mode (DIA); c) A pre-separation of peptide mixtures, for example, by HPLC (e.g., nano-UHPLC run with a gradient of acetonitrile in water) before or directly coupled to the mass spectrometry instrument; d) Replicate measurements of the same peptide mixture in order to allow a more robust statistical evaluation; e) A search of MS/MS spectra using different search engines (e.g., MASCOT, Sequest, Andromeda, XTandem, MS-GF+) or software tools using one of these search engines as well as de-novo sequence identification algorithms; f) A search of MS/MS spectra against different protein sequence databases (e.g. UniProtKB, IPI) as well as custom sequence databases generated for specific purposes (e.g., protein sequences translated from mRNA sequences); g) Mass spectrometry measurements of synthetic versions of the peptides in question to confirm their identity by comparing peptide specific characteristics, such as their MS/MS spectra and their retention time, for example, on an HPLC column; and h) A quantitative assessment of peptide signal areas for example by extraction and integration of MS1 features using appropriate algorithms. See, e.g., SuperHirn; Mueller et al. Proteomics (2007) 7: 3470-80.

In an aspect, the present disclosure relates to a method including isolating HLA peptides using chromatography, such as affinity chromatography. The isolated HLA ligands can be separated according to their hydrophobicity by reversed-phase chromatography (e.g., nanoAcquity UPLC system, Waters) followed by detection in an Orbitrap hybrid mass spectrometer (ThermoElectron). Each sample is preferably analyzed by acquisition of replicate, e.g., LC-MS runs. The LC-MS data is then processed by analyzing the Tandem-MS (MS/MS) data.

The MS/MS spectra recorded in a targeted way focusing on the m/z values of the peptides to be quantified may be evaluated preferably by a software that extracts the intensities of pre-selected fragment ions of pre-defined transitions. One example of such a software is Skyline, an application for analyzing mass spectrometer data of data independent acquisition (DIA) experiments for parallel reaction monitoring (PRM-targeted MS/MS). Information on Skyline is available, e.g., MacLean et al. “Skyline: an open source document editor for creating and analyzing targeted proteomics experiments.” Bioinformatics (2010) 26(7): 966-8.

Comparability of peptide groups restricted to the same HLA allele between different samples is possible based on a common allele-specific antibody used for purification, if available, or alternatively based on assignment of sequences to common HLA-alleles by means of anchor amino acid patterns.

### EXAMPLES

### Example 1

### HLA Peptidomic Data Generation

### Tissue Samples

Patients' tumor and normal tissues were provided by several different hospitals depending on the tumor entity analyzed. Written informed consents of all patients had been given before surgery. Tissues were shock-frozen in liquid nitrogen immediately after surgery and stored until isolation of HLA peptides at −80° C.

Isolation of HLA Peptides from Tissue Samples

HLA peptide pools from shock-frozen tissue samples were obtained by immune precipitation from solid tissues according to a slightly modified protocol using the HLA-A, -B, -C-specific antibody W6/32, the HLA-A*02-specific antibody BB7.2, CNBr-activated sepharose, acid treatment, and ultrafiltration. For different HLA-alleles other specific antibodies available in the art can be used as there are for example GAP-A3 for A*03, B1.23.2 for B-alleles.

### Mass Spectrometry

Mass spectrometry was performed according to the methods described in, for example, Zhang et al. (2018) Nature Communications 9: 3919; U.S. Provisional Patent Application No. 62/711,175; WO 2020/023845; U.S. Patent Application Publication No. 2016/0187351; U.S. Pat. Nos. 7,811,828; 9,783,849; 9,791,443; and 9,791,444, the contents of each which are herein incorporated by reference in their entirety. Briefly, peptides were eluted from antibody-resin by acid treatment and purified by ultrafiltration. For further separation, reversed-phase chromatography (nanoAcquity UPLC® system (a direct (non-split) capillary and nanoflow rates for high-resolution chromatographic separations) Waters, Milford, Mass.) was used eluting with an ACQUITY UPLC® BEH C18 column (75 μm×250 mm, Waters, Milford, Mass.) at a 190 min gradient ranging from 1 to 34.5% ACN. Eluted peptides were analysed by data-dependent acquisition (DDA) in an Orbitrap® mass spectrometer (Thermo Fisher Scientific, Waltham, Mass.) equipped with a nano electrospray ionisation (ESI) source. A total of 7825 runs was acquired in profile mode covering most samples with five replicate injections making use of different mass analysers in low- (TOP3, ion trap acquiring top 3 precursors) and high-resolution mode (TOP5, Orbitrap® acquiring top 5 precursors, R=7500), as well as different fragmentations using collision-induced dissociation (CID) and higher-energy collisional dissociation (HCD). Survey scans were acquired with high mass accuracy in the Orbitrap® (R=30,000 for TOP3, R=60,000 for TOP5). Mass range for selection of doubly charged precursors was 400-750 m/z and 800-1500 m/z for singly charged precursors. Spectra were extracted and centroided using Proteome Discoverer 1.4 (Thermo Fisher Scientific, Waltham, Mass.).

### Data Analysis

To generate peptide spectrum matches (PSMs), as shown in FIG. 12, experimental MS/MS spectra were analyzed by three database search engines including X! Tandem (Craig et al. (2004) J Proteome Res. 3:1234-1242), Comet (Eng et al. (2013) Proteomics. 13: 22-24), and MSGF+ (Kim et al. (2010) Mol Cell Proteomics. 9: 2840-2852) against Ensembl human proteome sequences. Search results from individual search engines were further analyzed by PeptideProphet (Keller et al. (2002) Anal Chem. 74: 5383-5392) for PSM validation and combined using iProphet algorithm (Shteynberg et al. (2011) Mol Cell Proteomics. 10: M111 007690). False discovery rate (FDR) was estimated using target-decoy approach (Elias et al. (2010) Methods Mol Biol. 604:55-71) based on iProphet probability scores. The contents of the references cited herein are incorporated by reference in their entireties. The system and methods described herein showed an unexpected improvement in generating predicted spectrums.

### Example 2

### Fragmentation Models

### Model Architecture

In an aspect, the peptide encoder includes three layers: (1) a bi-directional recurrent neural network (BDN) with gated recurrent memory units (GRU), (2) a recurrent GRU layer, and (3) an attention layer all with dropout. The recurrent layers use 512 memory cells each. The latent space is 512-dimensional. Precursor charge and NCE encoder is a single dense layer with the same output size as the peptide encoder. The latent peptide vector is decorated with the precursor charge and normalized collision energy (NCE) vector by element-wise multiplication. A one-layer length 29 BDN with GRUs, dropout and attention acts as decoder for fragment intensity. Implementation was done in Python with keras 2.1.1 and tensorflow 1.4.0 compiled to use GPUs.

### Training Data and Testing Data

In this example, inputs to the fragmentation models are, peptide sequences, precursor charge, and NCE. Peptide sequences are represented as discrete integer vectors of length 30, with each non-zero integer mapping to one amino acid and padded with zeros for sequences shorter than 30 amino acids. Precursor charge is one-hot encoded and NCE is normalized to [0, 1].

### Tryptic Peptide (PT) Data

ProteomeTools Dataset (Zolg et al. (2017) Nat Methods 14: 259-262, the content of which is incorporated by reference in its entirety) was downloaded from ProteomeXchange website via identifier PXD004732. ProteomeTools Dataset (PT) contains spectra from >330,000 synthetic tryptic peptides generated by various fragmentation techniques including HCD, CID, ETD, EThcD, and ETciD. Total 11.3×10⁶ PSMs corresponding to 211,000 peptide ions were analyzed by MaxQuant/Andromeda. For filtering, PSMs with PEP<0.05 and Andromeda score>100 were included. For each peptide ion group, only top 20 PSMs (Andromeda score) were selected. For CID 35 (30% NCE) fragmentation, the training data contains 717,355 PSMs corresponding to 158,952 peptide ions; and the testing data contains 75,977 PSMs corresponding to 17,661 peptide ions. For HCD 28 (28% NCE) fragmentation, the training data contains 778,276 PSMs corresponding to 174,420 peptide ions; and the testing data contains 86,458 PSMs corresponding to 19,380 peptide ions.

Use of HLA peptidomic (IM) data generated by following the methodology described in FIG. 12 (with pDeep algorithm).

All PSMs with run level false discovery rate (FDR)<0.05 are grouped into peptide ion group (Peptide sequence, charge state, and modification). For each peptide ion group, only top 20 PSMs based on iProphet probability were selected. For CID fragmentation 35% NCE (Normalized collision energy), the training data contains 2,569,200 PSMs corresponding to 559,395 peptide ions; and the testing data contains 900,124 PSMs corresponding to 41,573 peptide ions. For HCD 28% NCE fragmentation, the training data contains 2,103,904 PSMs corresponding to 351,247 peptide ions; and the testing data contains 462,668 PSMs corresponding to 36,715 peptide ions.

### Training

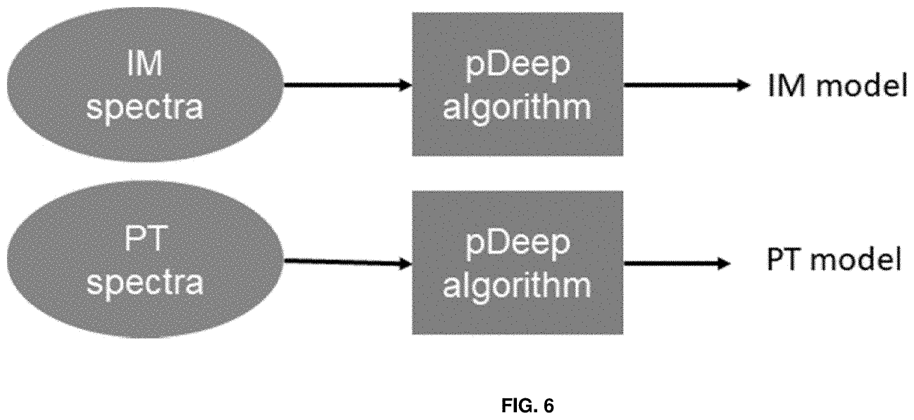

pDeep is a deep neural network-based model for the spectrum prediction of peptides. See, for example, Zhou et al. (2017) Anal. Chem. 89: 12690-1269). pDeep is based on the bidirectional long short-term memory (BiLSTM) and it is compatible for different fragmentation methods including higher-energy collisional dissociation, electron-transfer dissociation, and electron-transfer and higher-energy collision dissociation MS/MS spectra of peptides with prediction performance>0.9 median Pearson correlation coefficients. In addition, intermediate layer of the neural network can reveal physicochemical properties of amino acids, for example, the similarities of fragmentation behaviors between amino acids. FIG. 6 shows training data from the IM HLA peptidomic data (IM spectra developed by methods described herein) and ProteomeTools Dataset (PT spectra) that were input into pDeep algorithms to generate IM model (IM model was generated, for example, by following the protocol of FIG. 12 using pDeep) and PT model (ProteomeTools), respectively.

The ProteomeTools (PT model) was constructed using a dataset of 330,000 synthesized tryptic peptides. See Zolg et al. “Building ProteomeTools based on a complete synthetic human proteome,” Nature Methods (2017), the disclosure of which is hereby incorporated by reference in its entirety. Downloaded from ProteomeXchange (PXD004732) including data for more than 330,000 synthetic tryptic peptides (HCD, CID, ETD, ETHCD, ETCID). The data was Analyzed by MaxQuant/Andromeda, in total 11 3 million PSM (peptide spectrum match) yielding 211 thousand peptides. For filtering, PSMs with PEP less than 0.05 and Andromeda score larger than 100 were included. For each peptide ion group, only top 20 PSMs (Andromeda score) were selected. For CID (Collision-induced dissociation) 35 spectra set, the training data has 717,355 PSMs, which corresponds to 158,952 peptide ions, and the testing data has 75,977 PSMs, which corresponds to 17,661 peptide ions. For HCD (Higher-energy collisional dissociation) 28 spectra set, the training data has 778,276 PSMs, which corresponds to 174,420 peptide ions, and the testing data has 86,458 PSMs, which corresponds to 19,380 peptide ions.

The IM Model was constructed using 11,413 Tumor sample runs, 10,176 normal tissue runs, and 300,630,135 spectra. The tumors were from 20 major indications and the control tissues were from 40 normal tissues. PSMs with run level FDR<0.05. For each peptide ion group, only top 20 PSMs (iProphet probability) were selected. For CID 35 spectra set, the training data has 2,569,200 PSMs, which corresponds to 559,395 peptide ions, and the testing has 900,124 PSMs, which corresponds to 41,573 peptide ions. For HCD 28 spectra set, the training data has 2,103,904 PSMs, which corresponds to 351,247 peptide ions, and the testing data has 462,668 PSMs, which corresponds to 36,715 peptide ions.

### Prediction Performance Between IM Model and PT Model

FIG. 4 shows experimental spectra of peptide A (YLLPAIVHI; SEQ ID NO: 2) generated by MS/MS can be compared with the predicted spectra generated by a prediction model, e.g., pDeep model, trained by either IM spectra data (for example, as prepared in FIG. 12) or PT spectra data, for the identification of peptide A. Spectra similarities between the peptide A ion group are measured as technical variation.

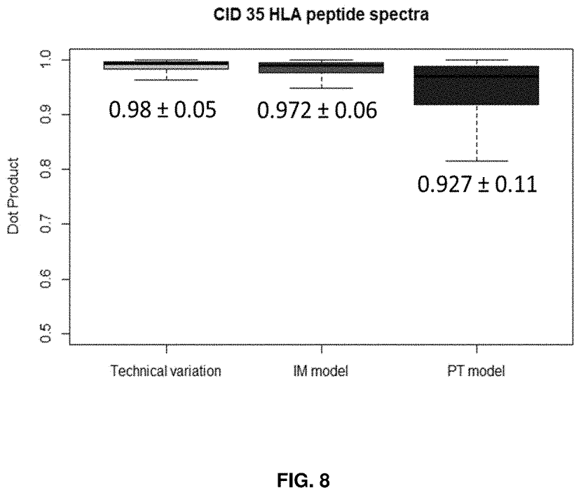

FIG. 8 shows IM prediction model achieves a better correlation (0.972±0.06) between testing spectra of CID 35 HLA peptides and the predicted spectra than PT model (0.927±0.11), as also indicated by the closer correlation of the former (99.2%) to the technical variation (0.98±0.05, 100%) than that of the latter (94.6%). This demonstrates that the pDeep machine learning system provided superior results.

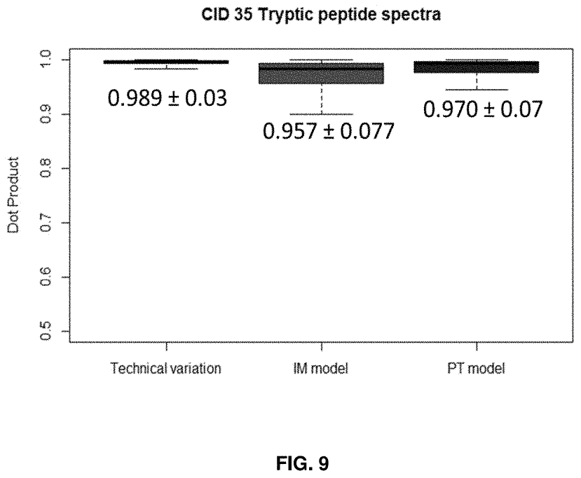

FIG. 9 shows that the PT prediction model achieved a correlation of (0.970±0.07) between testing spectra of CID 35 tryptic peptides and the IM model achieved a correlation of (0.957±0.077). This demonstrates that the pDeep machine learning system provided superior results.

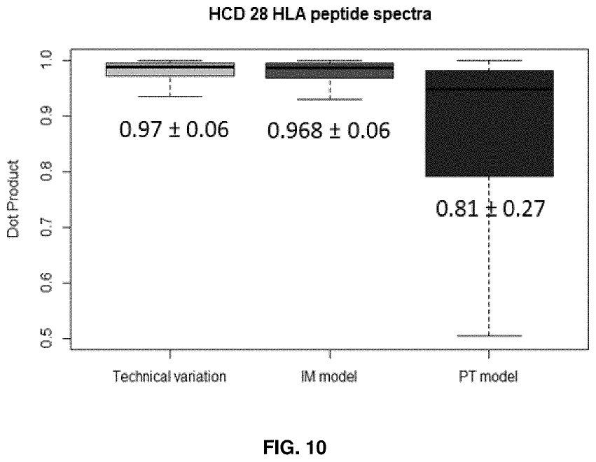

FIG. 10 shows IM prediction model achieves a better correlation (0.968±0.06) between testing spectra of HCD 28 HLA peptides and the predicted spectra than PT model (0.81±0.27), as also indicated by the closer correlation of the former (99.8%) to the technical variation (0.97±0.06, 100%) than that of the latter (83.5%). This demonstrates that the pDeep machine learning system provided superior results.

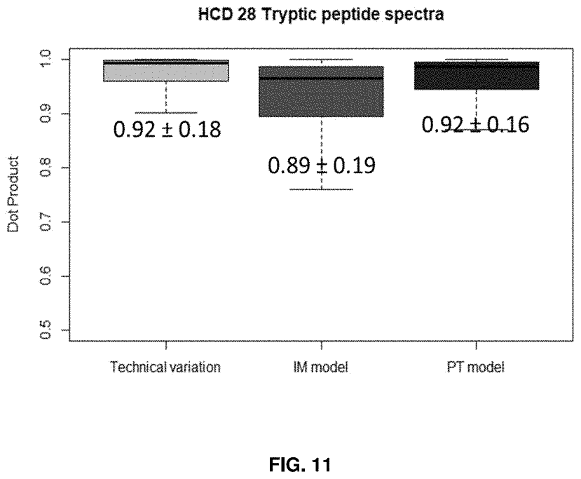

FIG. 11 shows PT prediction model achieves a correlation of (0.92±0.16) between testing spectra of HCD 28 tryptic peptides and the predicted spectra from the IM model achieves (0.89±0.19). The system and methods described herein showed an unexpected improvement in generating predicted spectrums.

### Example 3

### IM Model Performance

The IM model described herein was tested using peptides that are difficult to distinguish from one another and have a high false positive rate when tested using other models.

The IM Model used was constructed using Filtering criteria: PSMs with run level FDR<0.01, DeltaXC>0.1; Collision-induced dissociation (CID) fragmentation 35: Training data: 180,000 unique peptides; and Higher-energy collisional dissociation (HCD) fragmentation 25-27: Training data: 166,000 unique peptides. The IM model was compared the Prosit pretrained model (HCD 25) and Posit pretrain model (HCD 27). One limitation of Prosit is that it only provides prediction model for HCD spectra, but the system and methods described herein have both CID and HCD models. Therefore, the comparison was done only for HCD model.

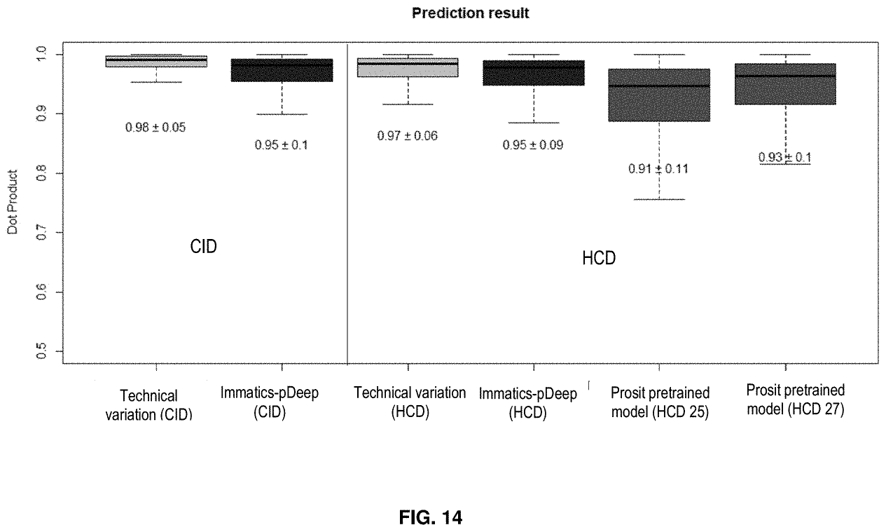

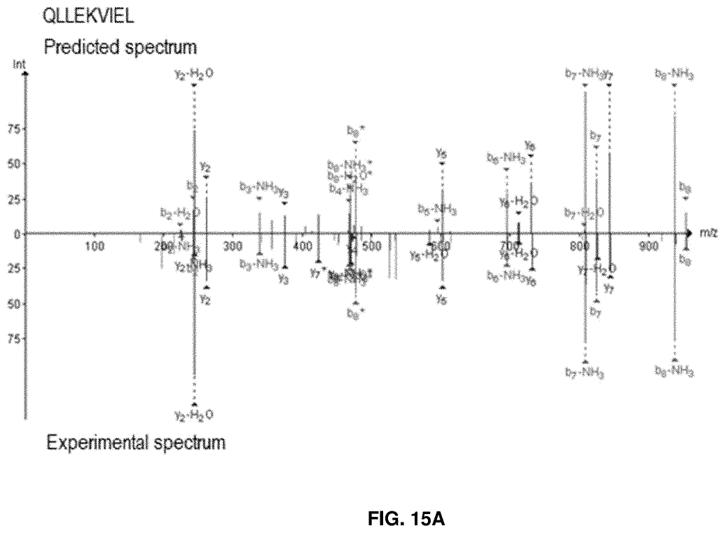

The Dot Product score we derived from Immatics-pDeep HCD model (an embodiment of the system and method described herein) was higher than Prosit's models, meaning that the spectra predicted by Immatics-pDeep HCD model is more similar to the spectra observed experimentally. See FIG. 14.

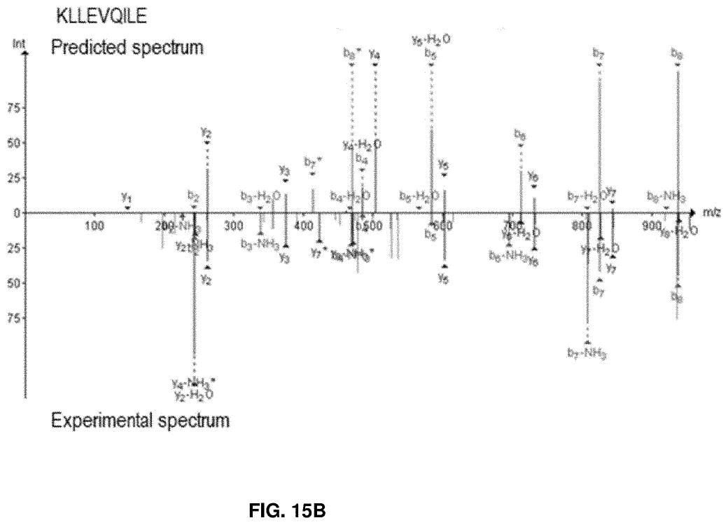

The inventors also surprisingly found that the Immatics-pDeep HCD model (an embodiment of the system and method described herein) was able to distinguish between two very similar peptides: KLLEVQILE (SEQ ID NO: 17) and QLLEKVIEL (SEQ ID NO: 18) which are difficult to distinguish using conventional methods. See FIG. 15. Initial mass spectrometry data analysis using conventional database search was not able to provide a clear decision between the two peptides QLLEKVIEL (SEQ ID NO: 18) and KLLEVQILE (SEQ ID NO: 17). Both peptides had similar XC (cross-correlation) scores computed by SEQUEST search engine. Using the Immatics-pDeep HCD model (an embodiment of the system and method described herein), the experimental spectrum was compared to the predicted spectra from the two peptides and spectral similarity computed using Dot Product. The inventors surprising found that the Dot Product computed by the predicted spectrum QLLEKVIEL is 0.927, much higher than the one computed by the other peptide. This data indicates that the Immatics-pDeep HCD model (an embodiment of the system and method described herein) was able to better distinguish peptides and therefore provide better peptide identification than conventional methodology.

To further investigate it in a larger dataset, 485 peptide pairs of ambiguous IDs from a database were selected. Similarly, one peptide in each pair was identified as the true peptide based on HLA binder score. The DotProduct scores computed by the true peptides are higher than false peptide in almost all the cases, showing again that the Immatics-pDeep HCD model (an embodiment of the system and method described herein) unexpected an improved peptide identification not just for one particular peptide pair, but for many different HLA peptides.

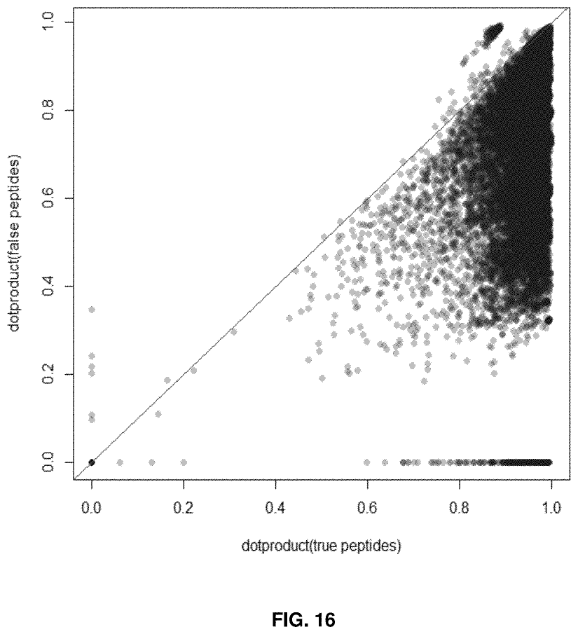

FIG. 16 depicts a Dot Product showing the Dot Product scores computed by the true peptides with high false peptide incidents, 485 peptide pairs where SEQUEST was not able to clearly differentiae one from the other. The Immatics-pDeep (HCD) (an embodiment of the system and method described herein) showed an unexpected improvement in the prediction of sequence for peptides that have a high incidence of false positives.

### Example 4

### Spectra Data and Retention Time

The inventors developed a rescoring algorithm that rescores that top 10 hits (peptide match) of each spectrum from Comet database search results. The cases that Comet search could not differentiate clearly, i.e., top N hits having similar Comet scores. The inventors used additional information from fragmentation and retention time predictions to better determine the accurate identification of the peptides.

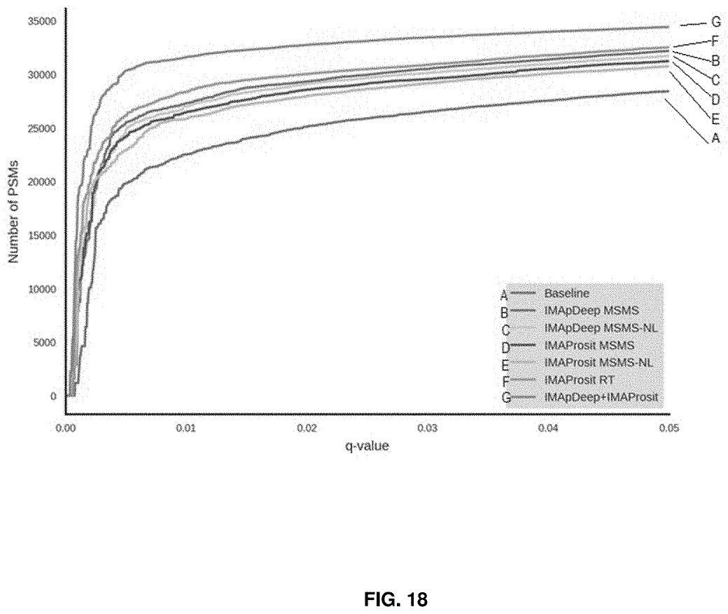

The dataset used for benchmarking the performance was HLA-B7+ LCL11 cell line DDA run. To compare among different approaches/models, the inventors used Percolator (Matthew The et al, Journal of the American Society for Mass Spectrometry, 28 Aug. 2016, 27(11):1719-1727) with target-decoy approach to estimate number of true hits given a fix q-value (False discovery rate). From the plot shown in FIG. 18, the inventors surprisingly discovered that IMApDeep+IMAProsit showed improved results. The inventors found they could identify 31,596 PSMs at 1% FDR compared to 22,519 PSMs in conventional Comet database search approach, posting an unexpected 40% improvement. Thus, the results suggest that the combination of fragmentation (spectra data) and retention time prediction can significantly improve MS/MS HLA peptide identification.

1. A method of identifying one of more antigenic peptides comprising:
a) obtaining one or more tissue samples,
b) acquiring mass spectrometry spectra for one or more antigenic peptides;
c) comparing the mass spectrometry data spectra to peptide theoretical spectra located in one or more public or non-public databases,
d) generating a peptide spectrum match (PSM) of the one or more antigenic peptides,
e) producing a matched spectral library or database of antigenic peptides based on steps (a)-(d),
f) using a deep learning algorithm to train at least 80% of the peptide data located in the database or spectral library and testing the balance of the peptide data located in the database or library thereby producing a peptide prediction model to generate predicted peptide spectrum;
g) using the prediction model to identify one or more antigenic peptides.

2. The method of claim 1, wherein the mass spectrometry comprises tandem mass spectrometry (MS/MS).

3. The method of claim 1, wherein the library or database comprises over about 70%, over about 80%, over about 85%, over about 90%, over about 95%, or 100% antigenic peptide data.

4. The method of claim 1, wherein the library or database comprises less than about 30%, less than about 25%, less than about 20%, less than about 15%, less than about 10%, or less than about 5% tryptic peptides data.

5. The method of claim 1, wherein the one or more antigenic peptides identified by the predicted spectra have an identification correlation within about 2% to about 15% relative to the actual technical variation of the experimentally determined spectra.

6. The method of claim 1, wherein the prediction peptide performance score is greater than about 0.95.

7. The method of claim 1, wherein the prediction peptide performance score is from about 0.92 to about 0.98.

8. The method of claim 6, wherein the peptide spectrum match (PSM) have a false discovery rate (FDR) of less than 0.05.

9. The method of claim 1, wherein antigenic peptides are identified with greater accuracy than tryptic peptides.

10. The method of claim 1, wherein the one or more identified antigenic peptides exhibit a peptide performance score that is closer to the measured technical variation as compared to analyzing the same one or more peptides with ProteomeTools.

11. The method of claim 1, wherein the antigenic peptides are 8 to 11 amino acid or 8 to 9 amino acids in length.

12. The method of claim 11, wherein the one or more antigenic peptides identified are overexpressed or presented in one or more specific cancer tissues.

13. The method of claim 12, wherein the tissue is a cancer tissue and is selected from the group consisting hepatocellular carcinoma (HCC), colorectal carcinoma (CRC), glioblastoma (GB), gastric cancer (GC), esophageal cancer, non-small cell lung cancer (NSCLC), pancreatic cancer (PC), renal cell carcinoma (RCC), benign prostate hyperplasia (BPH), prostate cancer (PCA), ovarian cancer (OC), melanoma, breast cancer (BRCA), chronic lymphocytic leukemia (CLL), Merkel cell carcinoma (MCC), small cell lung cancer (SCLC), Non-Hodgkin lymphoma (NHL), acute myeloid leukemia (AML), gallbladder cancer and cholangiocarcinoma (GBC, CCC), urinary bladder cancer (UBC), uterine cancer (UEC), and combination thereof.

14. The method of claim 11, wherein the spectral library or database comprises peptide data evaluated from over about 1500, over about 2000, over about 2500, or over about 3000 tissue samples.

15. The method of claim 14, wherein the spectral library or database comprises over about 100 million, over about 150 million, over about 180 million, or over about 200 million MS/MS spectra.

16. (canceled)

17. (canceled)

18. The method of claim 1, wherein the deep learning algorithm is selected from the group of pDeep, DeepMass, or PROSIT.

19. (canceled)

20. (canceled)

21. (canceled)

22. (canceled)

23. The method of claim 1, wherein the method further comprises
(a) acquiring retention time data for one or more antigenic peptides;
(b) comparing peptide retention time data to theoretical peptide retention time data in one or more public or non-public databases;
(c) generating a peptide spectrum match (PSM) of the one or more antigenic peptides using the retention time data,
(d) producing a matched spectral library or database of antigenic peptides based on steps (a)-(c),
(e) using a deep learning algorithm to train at least 80% of the peptide data located in the database or spectral library and testing the balance of the peptide data located in the database or library thereby producing a peptide prediction model to generate predicted peptide spectrum; and
(f) using the prediction model to identify one or more antigenic peptides.

24. A method classifying test data, the test data comprising peptide spectrum data, the method comprising:
(a) receiving, on at least one processor, test data comprising peptide spectrum data,
(b) evaluating, using the at least one processor, the test data using a classifier which is an electronic representation of a classification system, each said classifier trained using an electronically stored set of training data vectors, each training data vector representing an individual peptide and comprising a peptide spectrum data for the peptide, each training data vector further comprising a classification with respect to whether or not the peptide is antigenic,
(c) outputting, using the at least one processor, a classification of the sample from the peptide spectrum data concerning the likelihood of whether or not the peptide is antigenic based on the evaluating step.

25. A method of classifying test data, the test data comprising peptide spectra data, the method comprising:
(a) accessing, using at least one processor, an electronically stored set of training data vectors, each training data vector representing an individual cancer patient and comprising a peptide spectrum data for the respective cancer patient, each training data vector further comprising a classification with respect to whether or not a peptide is antigenic;
(b) training an electronic representation of a classification system, using the electronically stored set of training data vectors;
(c) receiving, at the at least one processor, test data comprising peptide spectrum data;
(d) evaluating, using the at least one processor, the test data using the electronic representation of the classification system; and
(e) outputting a classification of the test data concerning whether or not the peptide is antigenic based on the evaluating step.

26-80. (canceled)


SEQUENCE LISTING (18 sequences)

SEQ ID NO: 1 - protein, 8 aa, Artificial Sequence
  HLA peptide
APNDFNLK

SEQ ID NO: 2 - protein, 9 aa, Artificial Sequence
  HLA peptide
YLLPAIVHI

SEQ ID NO: 3 - protein, 9 aa, Artificial Sequence
  Target sequence
ISLLDAQSR

SEQ ID NO: 4 - protein, 10 aa, Artificial Sequence
  Target sequence
VVEELCPTPE

SEQ ID NO: 5 - protein, 8 aa, Artificial Sequence
  Target sequence
LLLQWCWE

SEQ ID NO: 6 - protein, 8 aa, Artificial Sequence
  Target sequence
CDVVSNTI

SEQ ID NO: 7 - protein, 9 aa, Artificial Sequence
  Decoy sequence
RSQADLLSI

SEQ ID NO: 8 - protein, 10 aa, Artificial Sequence
  Decoy sequence
EPTPCLEEVV

SEQ ID NO: 9 - protein, 8 aa, Artificial Sequence
  Decoy sequence
EWCWQLLL

SEQ ID NO: 10 - protein, 8 aa, Artificial Sequence
  Decoy sequence
ITNSVVDC

SEQ ID NO: 11 - protein, 9 aa, Artificial Sequence
  Peptide match
VSVVDLTNT

SEQ ID NO: 12 - protein, 9 aa, Artificial Sequence
  Peptide match
VVEELCEGM

SEQ ID NO: 13 - protein, 10 aa, Artificial Sequence
  Peptide match
DLLLQWCWEN

SEQ ID NO: 14 - protein, 10 aa, Artificial Sequence
  Peptide match
ECDVVTIIAE

SEQ ID NO: 15 - protein, 9 aa, Artificial Sequence
  Peptide match
GDAVIDALN

SEQ ID NO: 16 - protein, 9 aa, Artificial Sequence
  Peptide match
SYLFCMEAE

SEQ ID NO: 17 - protein, 9 aa, Artificial Sequence
  HLA peptide
KLLEVQILE

SEQ ID NO: 18 - protein, 9 aa, Artificial Sequence
  HLA peptide
QLLEKVIEL